(19) United States
(12) Patent Application Publication
(10) Pub. No.: US 2016/0287599 A1
(43) Pub. Date: Oct. 6, 2016

(54) SUBSTITUTED 1,2,4-TRIAZINE-3,5-DIONES AND THE USE THEREOF AS CHYMASE INHIBITORS

(71) Applicant: BAYER PHARMA AKTIENGESELLSCHAFT, Berlin (DE)

(72) Inventors: Chantal FÜRSTNER, Mülheim/Ruhr (DE); Jens ACKERSTAFF, Düsseldorf (DE); Alexander STRAUB, Wuppertal (DE); Heinrich MEIER, Wuppertal (DE); Hanna TINEL, Wuppertal (DE); Katja ZIMMERMANN, Düsseldorf (DE); Dmitry ZUBOV, Remscheid (DE); Jens SCHAMBERGER, Velbert - Langenberg (DE)

(73) Assignee: Bayer Pharma Aktiengesellschaft, Berlin (DE)

(21) Appl. No.: 15/035,171

(22) PCT Filed: Nov. 5, 2014
(86) PCT No.: PCT/EP2014/073799

§ 371 Date: May 6, 2016

(30) Foreign Application Priority Data
Nov. 8, 2013 (EP) 13192182.7

(51) Int. Cl. A61K 31/53; C07D 413/10; C07D 413/04; C07D 403/10; C07D 403/04; C07D 417/04; A61K 45/06; C07D 253/075

(52) U.S. Cl. CPC A61K 31/53; A61K 45/06; C07D 413/10; C07D 253/075; C07D 403/10; C07D 403/04; C07D 417/04; C07D 413/04

(57) ABSTRACT

Substituted 1,2,4-triazine-3,5-dione derivatives, processes for their preparation, their use alone or in combinations for the treatment and/or prophylaxis of diseases, and their use for preparing medicaments for the treatment and/or prophylaxis of diseases.

The present application relates to novel substituted 1,2,4-triazine-3,5-dione derivatives, to processes for their preparation, to their use alone or in combinations for the treatment and/or prophylaxis of diseases, and to their use for preparing medicaments for the treatment and/or prophylaxis of diseases.

Chymase is a chymotrypsin-like serine protease which is stored as a macromolecular complex with heparin proteoglycans in secretory vesicles of mast cells. After activation of the mast cells, chymase is released into the extracellular matrix and activated.

Activated mast cells play an important role in healing wounds and in inflammation processes, for example fibrosis of wounds, angiogenesis and cardiac remodelling (Miyazaki et al., Pharmacol. Ther. 112 (2006), 668-676; Shiota et., J. Hypertens. 21 (2003), 1823-1825). An increase in the number of mast cells has been observed in the event of heart failure, myocardial infarction and ischaemia, in human atherosclerotic plaques and in abdominal aortic aneurysms (Kovanen et al., Circulation 92 (1995), 1084-1088; Libby and Shi, Circulation 115 (2007), 2555-2558; Bacani and Frishman, Cardiol. Rev. 14(4) (2006), 187-193). Chymase-positive mast cells can also play an important role in the vascular remodelling of the respiratory pathways in the event of asthma and chronic obstructive pulmonary disease. An increased number of mast cells has been found in endobronchial biopsies of asthma patients (Zanini et al., J. Allergy Clin. Immunol. 120 (2007), 329-333). Moreover, chymase is suspected of being partly responsible for the genesis of many renal disorders, such as diabetic nephropathy and polycystic kidney disease (Huang et al., J. Am. Soc. Nephrol. 14(7) (2003), 1738-1747; McPherson et al., J. Am. Soc. Nephrol. 15(2) (2004), 493-500).

Chymase is predominantly involved in the production of angiotensin II in the heart, in the artery wall and in the lung, whereas the angiotensin-converting enzyme is responsible for the formation of the peptide in the circulation system (Fleming I., Circ. Res. 98 (2006), 887-896). In addition, chymase cleaves a number of other substrates of pathological significance. Chymase leads to degradation of extracellular matrix proteins, such as fibronectin, procollagen and vitronectin, and to the breakoff of focal adhesions. It brings about activation and release of TGFβ from its latent form, which plays an important role in the genesis of cardiac hypertrophy and cardiac fibrosis. The enzyme has atherogenic action, by degrading apolipoproteins and preventing the absorption of cholesterol by HDL. The action of chymase leads to release and activation of the cytokine interleukin 1 with its pro-inflammatory properties. Furthermore, it contributes to production of endothelin 1 (Bacani and Frishman, Cardiol. Rev. 14(4) (2006), 187-193). An accumulation of chymase-positive mast cells has been found in biopsies of patients having atopic dermatitis, Crohn's disease, chronic hepatitis and hepatic cirrhosis, and also idiopathic interstitial pneumonia (Dogrell S. A., Expert Opin. Ther. Patents 18 (2008), 485-499).

The possibility of using chymase inhibitors for the treatment of different diseases has been demonstrated in numerous studies involving animal experimentation Inhibition of chymase can be useful for the treatment of myocardial infarction. Jin et al. (Pharmacol. Exp. Ther. 309 (2004), 409-417) showed that a ligature of the coronary artery in dogs led to ventricular arrhythmias and elevated production of angiotensin II and chymase activity in the heart. Intravenous administration of the chymase inhibitor TY-501076 reduced chymase activity and the angiotensin II concentration in the plasma, and suppressed the occurrence of arrhythmias. A positive effect of chymase inhibition was shown in an in vivo model for myocardial infarction in hamsters. Treatment of the animals with the chymase inhibitor BCEAB reduced chymase activity, improved haemodynamics and reduced mortality (Jin et al., Life Sci. 71 (2002), 437-446). In the cardiomyopathic Syrian hamster, where the number of mast cells in the heart is elevated, oral treatment of the animals with the chymase inhibitor reduced cardiac fibrosis by 50% (Takai et al., Jpn. J. Pharmacol. 86 (2001), 124-126). In a tachycardia-induced heart failure model in dogs, chymase inhibition with SUN-C82257 led to reduction in the number of mast cells and in fibrosis in the heart. In addition, the diastolic function of the heart was improved after the treatment (Matsumoto et al., Circulation 107 (2003), 2555-2558).

Inhibition of chymase thus constitutes an effective principle in the treatment of cardiovascular disorders, inflammation and allergic disorders, and various fibrotic disorders.

WO 2007/150011 and WO 2009/049112 disclose a process for preparing pyrimidinetriones with glycine substituents. WO 2008/056257 describes triazinediones as GABA-B receptor modulators for the treatment of CNS disorders, WO 2004/058270 describes triazinediones as P2X₇ antagonists and WO 2012/002096 describes triazinedione derivatives as herbicides. WO 2008/103277 discloses various nitrogen heterocycles for treatment of cancer. It was an object of the present invention to provide novel substances which act as inhibitors of chymase and are suitable as such for treatment and/or prophylaxis of disorders, especially cardiovascular disorders.

The present invention relates to compounds of the general formula (I)

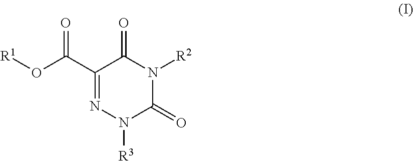

in which

R¹ represents hydrogen or (C₁-C₄)-alkyl,

R² represents a group of the formula

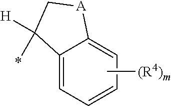

where

* represents the point of attachment to the triazinedione nitrogen atom,

A represents —CH₂—, —CH₂—CH₂—, —O—CH₂-** or oxygen, in which ** represents the point of attachment to the phenyl ring,

m represents a number 0, 1 or 2,

R⁴ represents hydrogen, halogen, difluoromethyl, trifluoromethyl, (C₁-C₄)-alkyl, difluoromethoxy, trifluoromethoxy or (C₁-C₄)-alkoxy,

R³ represents

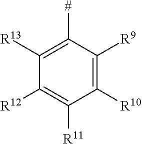

where

# represents the point of attachment to the triazinedione nitrogen atom,

R⁹ represents hydrogen,

R¹⁰ represents hydrogen, halogen, (C₁-C₄)-alkyl or (C₁-C₄)-alkoxy,

R¹¹ represents (C₁-C₄)alkyl, (C₁-C₄)-alkoxy or —N(R¹⁴R¹⁵),
in which (C₁-C₄)-alkyl may be up to trisubstituted by halogen,
in which (C₁-C₄)-alkoxy may be substituted by a substituent selected from the group consisting of hydroxy, (C₁-C₄)-alkoxycarbonyl, amino, mono-(C₁-C₄)-alkylamino, di-(C₁-C₄)-alkylamino, aminocarbonyl, mono-(C₁-C₄)-alkylaminocarbonyl and di-(C₁-C₄)-alkylaminocarbonyl,

where

R¹⁴ represents (C₁-C₄)-alkyl, (C₁-C₄)-alkoxycarbonyl or (C₁-C₄)-alkylaminocarbonyl,
in which (C₁-C₄)-alkylaminocarbonyl may be substituted by hydroxy or (C₁-C₄)-alkoxy,

R¹⁵ represents hydrogen or (C₁-C₄)-alkyl,

or

R¹¹ represents 4- to 7-membered heterocyclyl or 5- to 6-membered heteroaryl,
in which 4- to 7-membered heterocyclyl may be substituted by 1 to 3 substituents independently of one another selected from the group consisting of halogen, trifluoromethyl, (C₁-C₄)-alkyl, hydroxy, oxo, amino and (C₁-C₄)-alkoxycarbonyl,
in which 5- to 6-membered heteroaryl may be substituted by 1 or 2 substituents independently of one another selected from the group consisting of halogen, trifluoromethyl, (C₁-C₄)-alkyl, hydroxy, amino and (C₁-C₄)-alkoxycarbonyl,

R¹² represents hydrogen, halogen, cyano, (C₁-C₄)-alkyl or (C₁-C₄)-alkoxy,

R¹³ represents hydrogen, halogen, (C₁-C₄)-alkyl or (C₁-C₄)-alkoxy,

or

R³ represents

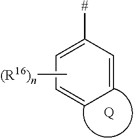

where

# represents the point of attachment to the triazinedione nitrogen atom,

the ring Q represents 5- to 7-membered heterocyclyl or 5- or 6-membered heteroaryl,
in which 5- to 7-membered heterocyclyl and 5- or 6-membered heteroaryl may be substituted by 1 to 4 substituents independently selected from the group of halogen, difluoromethyl, trifluoromethyl, trideuteromethyl, (C₁-C₆)-alkyl, (C₃-C₇)-cycloalkyl, oxo, hydroxyl, (C₁-C₄)-alkylcarbonyl, (C₁-C₄)-alkoxycarbonyl, aminocarbonyl and (C₁-C₄)-alkylsulfonyl,
in which (C₁-C₆)-alkyl and (C₃-C₇)-cycloalkyl may in turn be substituted by 1 to 3 substituents independently selected from the group of halogen, cyano, trifluoromethyl, (C₃-C₇)-cycloalkyl, hydroxyl, (C₁-C₄)-alkoxy and 4- to 7-membered heterocyclyl,
and
in which two (C₁-C₆)-alkyl radicals attached to a carbon atom of 5- to 7-membered heterocyclyl together with the carbon atom to which they are attached may form a 3- to 6-membered carbocycle,

R¹⁶ represents halogen, (C₁-C₄)-alkyl or (C₁-C₄)-alkoxy,

n represents a number 0, 1, 2 or 3,

and the salts, solvates and solvates of the salts thereof.

Compounds of the invention are the compounds of the formula (I) and the salts, solvates and solvates of the salts thereof, the compounds that are encompassed by formula (I) and are of the formulae given below and the salts, solvates and solvates of the salts thereof and the compounds that are encompassed by the formula (I) and are mentioned below as embodiments and the salts, solvates and solvates of the salts thereof if the compounds that are encompassed by the formula (I) and are mentioned below are not already salts, solvates and solvates of the salts.

The compounds of the invention may, depending on their structure, exist in different stereoisomeric forms, i.e. in the form of configurational isomers or else, if appropriate, of conformational isomers (enantiomers and/or diastereomers, including those in the case of atropisomers). The present invention therefore encompasses the enantiomers and diastereomers and the respective mixtures thereof. It is possible to isolate the stereoisomerically homogeneous constituents from such mixtures of enantiomers and/or diastereomers in a known manner.

If the compounds of the invention can occur in tautomeric forms, the present invention encompasses all the tautomeric forms.

Preferred salts in the context of the present invention are physiologically acceptable salts of the compounds according to the invention. Also encompassed are salts which are not themselves suitable for pharmaceutical applications but can be used, for example, for isolation or purification of the compounds of the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The present invention additionally also encompasses prodrugs of the compounds of the invention. The term “prodrugs” encompasses compounds which for their part may be biologically active or inactive but are converted during their residence time in the body into compounds according to the invention (for example by metabolism or hydrolysis).

In the context of the present invention, unless specified otherwise, the substituents are defined as follows:

Alkyl in the context of the invention is a straight-chain or branched alkyl radical having 1 to 4 carbon atoms. The following may be mentioned by way of example and by way of preference: methyl, ethyl, n-propyl, isopropyl, n-butyl, isobutyl, sec-butyl and tent-butyl.

Alkylcarbonyloxy in the context of the invention is a straight-chain or branched alkylcarbonyl radical which is attached via an oxygen atom and carries 1 to 4 carbon atoms in the alkyl chain. The following may be mentioned by way of example and by way of preference: methylcarbonyloxy, ethylcarbonyloxy, n-propylcarbonyloxy, isopropylcarbonyloxy, n-butylcarbonyloxy, isobutylcarbonyloxy and tert-butylcarbonyloxy.

Alkoxy in the context of the invention is a straight-chain or branched alkoxy radical 1 to 4 carbon atoms. The following may be mentioned by way of example and by way of preference: methoxy, ethoxy, n-propoxy, isopropoxy, n-butoxy and tert-butoxy.

Alkoxycarbonyl in the context of the invention is a straight-chain or branched alkoxy radical having 1 to 4 carbon atoms and a carbonyl group attached to the oxygen. Preference is given to a linear or branched alkoxycarbonyl radical having 1 to 4 carbon atoms in the alkoxy group. The following may be mentioned by way of example and by way of preference: methoxycarbonyl, ethoxycarbonyl, n-propoxycarbonyl, isopropoxycarbonyl and tert-butoxycarbonyl.

Alkoxycarbonylamino in the context of the invention is an amino group having a linear or branched alkoxycarbonyl substituent which has 1 to 4 carbon atoms in the alkyl chain and is attached to the nitrogen atom via the carbonyl group. The following may be mentioned by way of example and by way of preference: methoxycarbonylamino, ethoxycarbonylamino, propoxycarbonylamino, n-butoxycarbonylamino, isobutoxycarbonylamino and tent-butoxycarbonylamino

Alkylsulfonyl in the context of the invention is a straight-chain or branched alkyl radical which has 1 to 4 carbon atoms and is attached via a sulfonyl group. Preferred examples include: methylsulfonyl, ethylsulfonyl, n-propylsulfonyl, isopropylsulfonyl, n-butylsulfonyl and tert-butylsulfonyl.

Monoalkylamino in the context of the invention is an amino group having a linear or branched alkyl substituent having 1 to 4 carbon atoms. The following may be mentioned by way of example and by way of preference: methylamino, ethylamino, n-propylamino, isopropylamino and tert-butylamino.

Dialkylamino in the context of the invention is an amino group having two identical or different, straight-chain or branched alkyl substituents each having 1 to 4 carbon atoms. The following may be mentioned by way of example and by way of preference: N,N-dimethylamino, N,N-diethylamino, N-ethyl-N-methylamino, N-methyl-N-n-propylamino, N-isopropyl-N-n-propylamino and N-tert-butyl-N-methylamino.

Monoalkylaminocarbonyl in the context of the invention is an amino group which is attached via a carbonyl group and has a straight-chain or branched alkyl substituent having 1 to 4 carbon atoms.

The following may be mentioned by way of example and by way of preference: methylaminocarbonyl, ethylaminocarbonyl, n-propylaminocarbonyl, isopropylaminocarbonyl, n-butylaminocarbonyl and tert-butylaminocarbonyl.

Dialkylaminocarbonyl in the context of the invention is an amino group which is attached via a carbonyl group and has two identical or different, straight-chain or branched alkyl substituents each having 1 to 4 carbon atoms. The following may be mentioned by way of example and by way of preference: N,N-dimethylaminocarbonyl, N,N-diethylaminocarbonyl, N-ethyl-N-methylaminocarbonyl, N-methyl-N-n-propylaminocarbonyl, N-n-butyl-N-methylaminocarbonyl and N-tert-butyl-N-methylaminocarbonyl.

Monoalkylaminocarbonylamino in the context of the invention is an amino group which carries a straight-chain or branched alkylaminocarbonyl substituent having 1 to 4 carbon atoms in the alkyl chain and is attached via the carbonyl group. The following may be mentioned by way of example and by way of preference: methylaminocarbonylamino, ethylaminocarbonylamino, n-propylaminocarbonylamino, isopropylaminocarbonylamino, n-butylaminocarbonylamino and tert-butylaminocarbonylamino.

Dialkylaminocarbonylamino in the context of the invention is an amino group which carries a straight-chain or branched dialkylaminocarbonyl substituent having in each case 1 to 4 carbon atoms in the alkyl chain which may be identical or different, and is attached via the carbonyl group.

The following may be mentioned by way of example and by way of preference: N,N-dimethylaminocarbonylamino, N,N-diethylaminocarbonylamino, N-ethyl-N-methylaminocarbonylamino, N-methyl-N-n-propylaminocarbonylamino, N-n-butyl-N-methylaminocarbonylamino and N-tert-butyl-N-methylaminocarbonylamino

Heterocyclyl or heterocycle in the context of the invention is a saturated or partially unsaturated heterocycle having a total of 4 to 7 ring atoms which contains 1 to 3 ring heteroatoms from the group consisting of N, O and S and is attached via a ring carbon atom or optionally a ring nitrogen atom. Examples include: azetidinyl, pyrrolidinyl, tetrahydrofuranyl, imidazolidinyl, dihydroimidazolyl, pyrazolidinyl, dihydrotriazolyl, oxazolidinyl, dihydrooxazolyl, thiazolidinyl, dihydrooxadiazolyl, piperidinyl, piperazinyl, tetrahydropyranyl, oxazinanyl, hexahydropyrimidinyl, morpholinyl, thiomorpholinyl and azepanyl. Preference is given to 5- or 6-membered heterocyclyl radicals having 1 to 3 ring heteroatoms. The following may be mentioned by way of example and by way of preference imidazolidinyl, dihydroimidazolyl, pyrazolidinyl, dihydrotriazolyl, oxazolidinyl, dihydrooxazolyl, piperazinyl and morpholinyl.

Heteroaryl in the context of the invention is a monocyclic aromatic heterocycle (heteroaromatic) which has a total of 5 or 6 ring atoms, contains up to three identical or different ring heteroatoms from the group consiting of N, O and S and is joined via a ring carbon atom or via any ring nitrogen atom. Examples include: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl and triazinyl. Preference is given to monocyclic 5-membered heteroaryl radicals having two or three ring heteroatoms from the group consisting of N, O and S, for example thiazolyl, oxazolyl, isothiazolyl, isoxazolyl, pyrazolyl, imidazolyl, triazolyl, oxadiazolyl and thiadiazolyl.

Halogen in the context of the invention includes fluorine, chlorine, bromine and iodine. Preference is given to chlorine or fluorine.

An oxo group in the context of the invention is an oxygen atom attached to a carbon atom via a double bond.

In the formulae of the group that A, R², R³ and R¹¹ may represent, the end point of the line marked by a symbol * or ** or # or ## does not represent a carbon atom or a CH₂ group but is part of the bond to the respective atom to which A, R², R³ and R¹¹, respectively, are attached.

When radicals in the compounds of the invention are substituted, the radicals may be mono- or polysubstituted, unless specified otherwise. In the context of the present invention, all radicals which occur more than once are defined independently of one another. Substitution by one or two identical or different substituents is preferred. Very particular preference is given to substitution by one substituent.

Preference is given in the context of the present invention to compounds of the formula (I) in which

R¹ represents hydrogen or (C₁-C₄)-alkyl,

R² represents a group of the formula

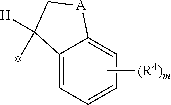

where
* represents the point of attachment to the triazinedione nitrogen atom,
A represents —CH₂— or —CH₂—CH₂—,
m represents a number 0, 1 or 2,
R⁴ represents hydrogen, fluorine, chlorine, difluoromethyl, trifluoromethyl or methyl,

R³ represents

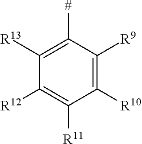

where
# represents the point of attachment to the triazinedione nitrogen atom,
R⁹ represents hydrogen,
R¹⁰ represents hydrogen, halogen or (C₁-C₄)-alkoxy,
R¹¹ represents (C₁-C₄)-alkyl, (C₁-C₄)-alkoxy or —N(R¹⁴R¹⁵),
   where
   R¹⁴ represents (C₁-C₄)-alkyl,
   R¹⁵ represents hydrogen or (C₁-C₄)-alkyl,
or
R¹¹ represents 5- or 6-membered heterocyclyl,
   in which 5- or 6-membered heterocyclyl may be substituted by 1 or 2 substituents independently of one another selected from the group consisting of trifluoromethyl, (C₁-C₄)-alkyl and oxo,
R¹² represents hydrogen,
R¹³ represents hydrogen or (C₁-C₄)-alkyl,

or

R³ represents a group of the formula

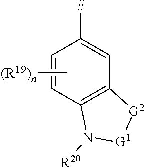

where
# represents the point of attachment to the triazinedione nitrogen atom,
G¹ represents C═O or SO₂,
G² represents CR^{21A}R^{21B}, NR²², O or S,
   where
   R^{21A} represents hydrogen, fluorine, (C₁-C₄)-alkyl or hydroxy,
   R^{21B} represents hydrogen, fluorine, chlorine, (C₁-C₄)-alkyl or trifluoromethyl, or
   R^{21A} and R^{21B} together with the carbon atom to which they are attached form a 3- to 6-membered carbocycle,
   R22 represents hydrogen, (C₁-C₆)-alkyl or (C₃-C₇)-cycloalkyl,
R¹⁹ represents fluorine or methyl,
n represents a number 0 or 1,
R20 represents hydrogen, (C₁-C₆)-alkyl or (C₃-C₆)-cycloalkyl,

and the salts, solvates and solvates of the salts thereof.

Particular preference is given in the context of the present invention to compounds of the formula (I) in which

R¹ represents hydrogen, methyl or ethyl,

R² represents a group of the formula

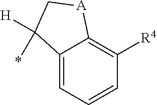

where
* represents the point of attachment to the triazinedione nitrogen atom,
A represents —CH₂— or —CH₂—CH₂—,
R⁴ represents chlorine or trifluoromethyl,

R³ represents

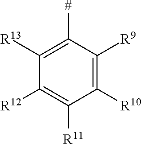

in which
# represents the point of attachment to the triazinedione nitrogen atom,
R⁹ represents hydrogen,
R¹⁰ represents hydrogen,
R¹¹ represents methoxy or ethoxy,
or
R¹¹ represents a group of the formula

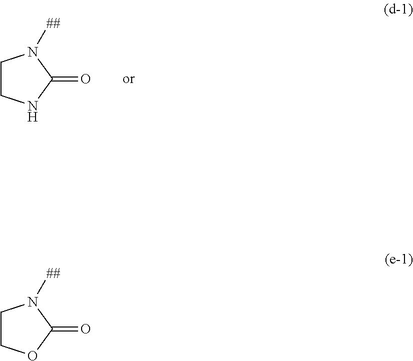

in which
   ## represents the point of attachment to the phenyl ring,
R¹² represents hydrogen,
R¹³ represents hydrogen or methyl,

or

R³ represents a group of the formula

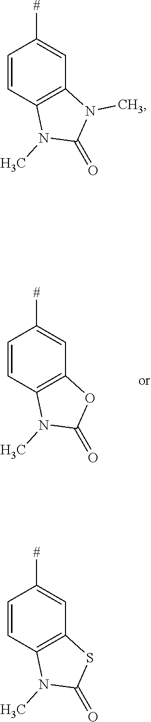

where
# represents the point of attachment to the triazinedione nitrogen atom,

and the salts, solvates and solvates of the salts thereof.

In the context of the present invention, preference is also given to compounds of the formula (I) in which

R² represents a group of the formula

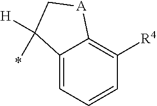

where
* represents the point of attachment to the triazinedione nitrogen atom,
A represents —CH₂— or —CH₂—CH₂—,
R⁴ represents chlorine or trifluoromethyl,

and the salts, solvates and solvates of the salts thereof.

In the context of the present invention, preference is also given to compounds of the formula (I) in which

R³ represents

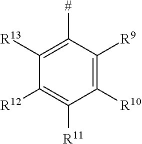

in which
# represents the point of attachment to the triazinedione nitrogen atom,
R⁹ represents hydrogen,
R¹⁰ represents hydrogen,
R¹¹ represents a group of the formula

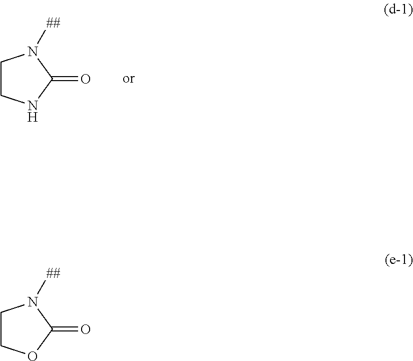

in which
   ## represents the point of attachment to the phenyl ring,
R¹² represents hydrogen,
R¹³ represents hydrogen,

and the salts, solvates and solvates of the salts thereof.

In the context of the present invention, preference is also given to compounds of the formula (I) in which

R³ represents a group of the formula

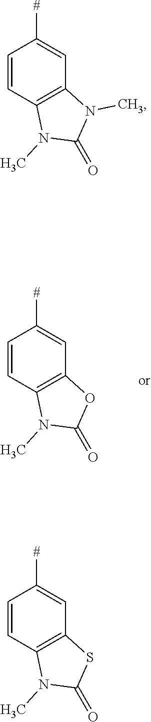

where
# represents the point of attachment to the triazinedione nitrogen atom,

and the salts, solvates and solvates of the salts thereof.

Irrespective of the particular combinations of the radicals specified, the individual radical definitions specified in the particular combinations or preferred combinations of radicals are also replaced as desired by radical definitions from other combinations.

Very particular preference is given to combinations of two or more of the abovementioned preferred ranges.

The invention further provides a process for preparing compounds of the formula (I) according to the invention, characterized in that

[A] a compound of the formula (II)

H₂N—R³ (II)

in which

R³ has the meaning given above,

is diazotized in an inert solvent using sodium nitrite and a suitable acid to give a compound of the formula (II-1)

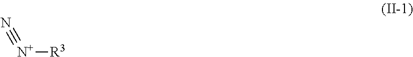

in which

R³ has the meaning given above,

and the diazonium salt is, optionally in the presence of a suitable base, reacted with a compound of the formula (III)

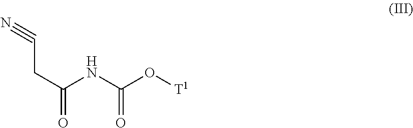

in which

T¹ represents (C₁-C₄)-alkyl,

to give a compound of the formula (IV)

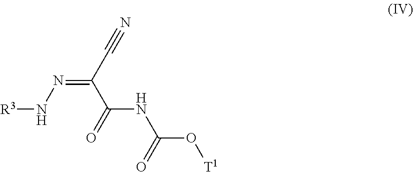

in which

R³ and T¹ each have the meanings given above,

this is then converted in an inert solvent, optionally in the presence of a suitable base, into a compound of the formula (V)

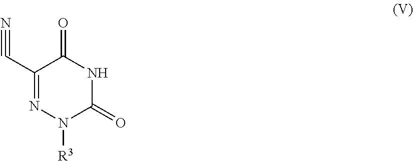

in which

R³ has the meaning given above,

subsequently reacted under Mitsunobu conditions with an activating agent, e.g. diethyl azodicarboxylate (DEAD) or diisopropyl azodicarboxylate (DIAD), and a phosphine reagent, e.g. triphenylphosphine or tributylphosphine, in an inert solvent with a compound of the formula (VI)

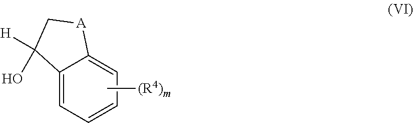

to give a compound of the formula (VII)

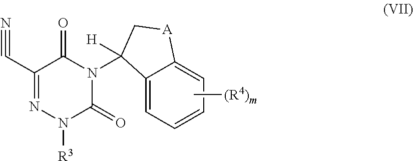

in which

A, m, R³ and R⁴ have the meanings given above,

and this is then hydrolyzed in an inert solvent in the presence of a suitable acid or base to give a compound of the formula (I-1)

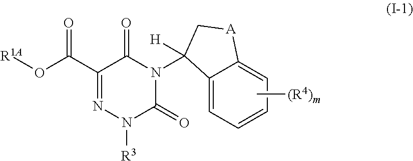

in which

A, m, R³ and R⁴ have the meanings given above, and

R^{1A} represents hydrogen,

or

[B] a compound of the formula (V)

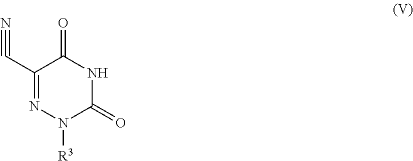

in which

R³ has the meaning given above,

is hydrolyzed in an inert solvent in the presence of a suitable acid or base to give a compound of the formula (VIII)

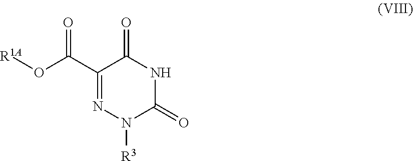

in which

R^{1A} represents hydrogen,

and

R³ has the meaning given above,

the acid function is then esterified to give a compound of the formula (IX)

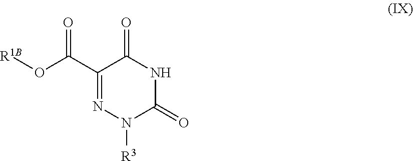

in which

R³ has the meanings given above,

and

R^{1B} represents (C₁-C₄)-alkyl,

and this is subsequently analogously to process [A] under Mitsunobu conditions with an activating agent, e.g. diethyl azodicarboxylate (DEAD) or diisopropyl azodicarboxylate (DIAD), and a phosphine reagent, e.g. triphenylphosphine or tributylphosphine, in an inert solvent with a compound of the formula (VI)

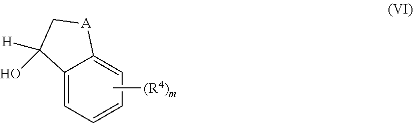

in which

A, m and R⁴ have the meanings given above,

converted into a compound of the formula (I-2)

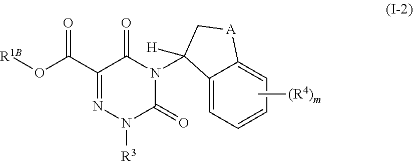

in which

A, m, R³ and R⁴ have the meanings given above,

and

R^{1B} represents (C₁-C₄)-alkyl,

or

[C] a compound of the formula (I-2) is hydrolysed in an inert solvent in the presence of a suitable acid or base to give a compound of the formula (I-1)

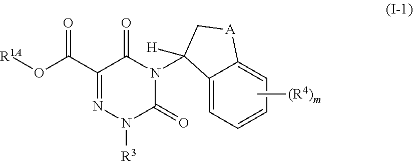

in which A, m, R³ and R⁴ each have the meanings given above,

and

R^{1A} represents hydrogen,

any protecting groups are detached and/or the compounds of the formulae (I-1) and (I-2) are, where appropriate, converted with the appropriate (i) solvents and/or (ii) bases or acids to the solvates, salts and/or solvates of the salts thereof.

The compounds of the formulae (I-1) and (I-2) together form the group of compounds of the formula (I) according to the invention.

Inert solvents for the process step (II)→(II-1) and (II-1)+(III)→(IV) are, for example, alcohols such as methanol, ethanol, n-propanol, isopropanol or n-butanol, or other solvents such as dimethylformamide, dimethyl sulfoxide, N,N′-dimethylpropyleneurea (DMPU), N-methylpyrrolidinone (NMP), pyridine, acetone, 2-butanone; sulfolane, sulfolene, water or acetonitrile. It is also possible to use mixtures of the solvents mentioned. Preference is given to using water.

Suitable acids for the process step (II)→(II-1) are, for example, hydrochloric acid, sulfuric acid, phosphoric acid or acetic acid. Preference is given to using hydrochloric acid.

Suitable bases for the process steps (II-1)+(III)→(IV) and (IV)→(V) are alkali metal alkoxides such as sodium methoxide or potassium methoxide, sodium ethoxide or potassium ethoxide or sodium tert-butoxide or potassium tert-butoxide, alkali metal carboxylates such as sodium acetate or potassium acetate, alkali metal hydrides such as sodium hydride or potassium hydride, amides such as sodium amide, lithium bis(trimethylsilyl)amide or potassium bis(trimethylsilyl)amide or lithium diisopropylamide, or organic bases such as pyridine, triethylamine, diisopropylethylamine, 1,5-diazabicyclo[4.3.0]non-5-ene (DBN), 1,8-diazabicyclo[5.4.0]undec-7-ene (DBU) or 1,4-diazabicyclo[2.2.2]octane (DABCO®) or phosphazene bases, for example 1-[N-tert-butyl-P,P-di(pyrrolidin-1-yl)phosphorimidoyl]pyrrolidine or N′″-tert-butyl-N,N,N′,N′-tetramethyl-N″-[tris(dimethylamino)-lambda⁵-phosphanylidene]phosphorimidetriamide. Preference is given to pyridine, sodium acetate, sodium ethoxide and potassium tert-butoxide.

The reaction (II)→(II-1) generally takes place in a temperature range from 0° C. to +30° C., preferably at 0° C. In general, the reaction is carried out at atmospheric pressure.

The reaction (II-1)+(III)→(IV) is generally carried out in a temperature range of from 0° C. to +150° C., preferably at +20° C. to +120° C. The reaction can be performed at atmospheric, elevated or reduced pressure (for example from 0.5 to 5 bar). In general, the reactions are carried out at atmospheric pressure.

The reactions (V)+(VI)→(VII) and (IX)+(VI)→(I-1) are carried out under Mitsunobu conditions [see: a) Hughes, D. L. “The Mitsunobu Reaction” Organic Reactions; John Wiley & Sons, Ltd, 1992, vol. 42, p. 335. b) Hughes, D. L. Org. Prep. Proceed. Int. 1996, 28, 127]. The Mitsunobu reaction is effected using triphenylphosphine, or tri-n-butylphosphine, 1,2-bis(diphenylphosphino)ethane (DPPE), diphenyl(2-pyridyl)phosphine (Ph2P-Py), (p-dimethylaminophenyl)diphenylphosphine (DAP-DP), tris(4-dimethylaminophenyl)phosphine (tris-DAP), and a suitable dialkyl azodicarboxylate, for example diethyl azodicarboxylate (DEAD), diisopropyl azodicarboxylate (DIAD), di-tert-butyl azodicarboxylate, N,N,N′N′-tetramethylazodicarboxamide (TMAD), 1,1′-(azodicarbonyl)dipiperidine (ADDP) or 4,7-dimethyl-3,5,7-hexahydro-1,2,4,7-tetrazocine-3,8-dione (DHTD). Preference is given to using triphenylphosphine and diisopropyl azodicarboxylate (DIAD).

Inert solvents for the Mitsunobu reactions (V)+(VI)→(VII) and (IX)+(VI)→(I-1) are, for example, ethers such as tetrahydrofuran, diethyl ether, hydrocarbons such as benzene, toluene, xylene, halohydrocarbons such as dichloromethane, dichloroethane or other solvents such as acetonitrile or dimethylformamide (DMF). It is also possible to use mixtures of the solvents mentioned. Preference is given to using THF or a mixture of THF and DMF.

The Mitsunobu reactions (V)+(VI)→(VII) and (IX)+(VI)→(I-1) are generally carried out within a temperature range from −78° C. to +180° C., preferably at 0° C. to +50° C., optionally in a microwave. The conversions can be performed at atmospheric, elevated or reduced pressure (for example from 0.5 to 5 bar).

The hydrolysis of the nitrile group of the compounds (V) and (VII) to compounds of the formula (VIII) or (I-1) is carried out by treating the nitriles in inert solvents with suitable acids.

Suitable acids for the hydrolysis of the nitrile group are, in general, sulfuric acid, hydrogen chloride/hydrochloric acid, hydrogen bromide/hydrobromic acid, phosphoric acid or acetic acid or mixtures thereof, optionally with addition of water. Preference is given to hydrogen chloride.

Suitable inert solvents for these reactions are water, diethyl ether, tetrahydrofuran, dioxane or glycol dimethyl ether, or other solvents such as acetonitrile, acetic acid, dimethylformamide or dimethyl sulfoxide. It is also possible to use mixtures of the solvents mentioned. Preference is given to acetic acid.

The hydrolysis of the nitrile group generally takes place within a temperature range from 0° C. to 180° C., preferably at +80° C. to 120° C.

These conversions can be performed at atmospheric, elevated or reduced pressure (for example from 0.5 to 5 bar). In general, the reactions are in each case carried out at atmospheric pressure.

The esterification of the acid group R^{1A} of the compound (VIII) to give compounds of the formula (IX) is carried out by treating the acid in a suitable solvent with an alcohol, for example methanol or ethanol, in the presence of thionyl chloride.

Suitable solvents for this reaction are alcohols such as methanol, ethanol, n-propanol, isopropanol, n-butanol or tert-butanol, tetrahydrofuran, dioxane or glycol dimethyl ether, or other solvents such as acetonitrile, dimethylformamide or dimethyl sulfoxide. It is also possible to use mixtures of the solvents mentioned. The preferred solvent is the alcohol which participates in the reaction, for example methanol or ethanol.

Alternatively, the acid may first be converted with thionyl chloride into the acid chloride, which can then be reacted with an alcohol of the formula R^{1B}OH.

Alternatively, the esterification of the acid group R^{1A} of the compound (VIII) to give compounds of the formula (IX) can take place by heating the compound of the formula (VIII) with an alcohol of the formula R^{1B}OH in the presence of an inorganic acid such as hydrogen chloride, sulfuric acid or phosphoric acid.

The esterification is generally carried out within a temperature range from 0° C. to 180° C., preferably at +20° C. to 120° C.

These conversions can be performed at atmospheric, elevated or reduced pressure (for example from 0.5 to 5 bar). In general, the reactions are in each case carried out at atmospheric pressure.

The hydrolysis of the ester group R^{1A} of the compound (I-2) to compounds of the formula (I-1) is effected by treating the esters in inert solvents with acids or bases, in which latter case the salts formed at first are converted to the free carboxylic acids by treating with acid. In general, the ester hydrolysis is preferably effected with acids.

Suitable inert solvents for these reactions are water, diethyl ether, tetrahydrofuran, dioxane or glycol dimethyl ether, or other solvents such as acetonitrile, acetic acid, dimethylformamide or dimethyl sulfoxide. It is also possible to use mixtures of the solvents mentioned. In the case of a basic ester hydrolysis, preference is given to using mixtures of water with dioxane, tetrahydrofuran or acetonitrile. For the hydrolysis of tert-butyl esters, the solvent used in the case of reaction with trifluoroacetic acid is preferably dichloromethane, and in the case of reaction with hydrogen chloride preferably tetrahydrofuran, diethyl ether or dioxane. For the hydrolysis of other esters under acidic conditions, preference is given to acetic acid or a mixture of acetic acid and water.

Suitable bases are the alkali metal or alkaline earth metal hydrogencarbonates such as sodium or potassium hydrogencarbonate. Preference is given to sodium hydrogencarbonate.

Suitable acids for the ester cleavage are generally sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, optionally with addition of water. Preference is given to hydrogen chloride or trifluoroacetic acid in the case of the tert-butyl esters, and to hydrochloric acid in a mixture with acetic acid, and to sulfuric acid in a mixture with acetic acid and water in the case of the methyl esters and ethyl esters.

The ester hydrolysis is generally carried out within a temperature range from 0° C. to 180° C., preferably at +20° C. to 120° C.

These conversions can be performed at atmospheric, elevated or reduced pressure (for example from 0.5 to 5 bar). In general, the reactions are in each case carried out at atmospheric pressure.

The preparation of the compounds according to the invention can be illustrated by way of example by the following synthesis schemes (Schemes 1 and 2):

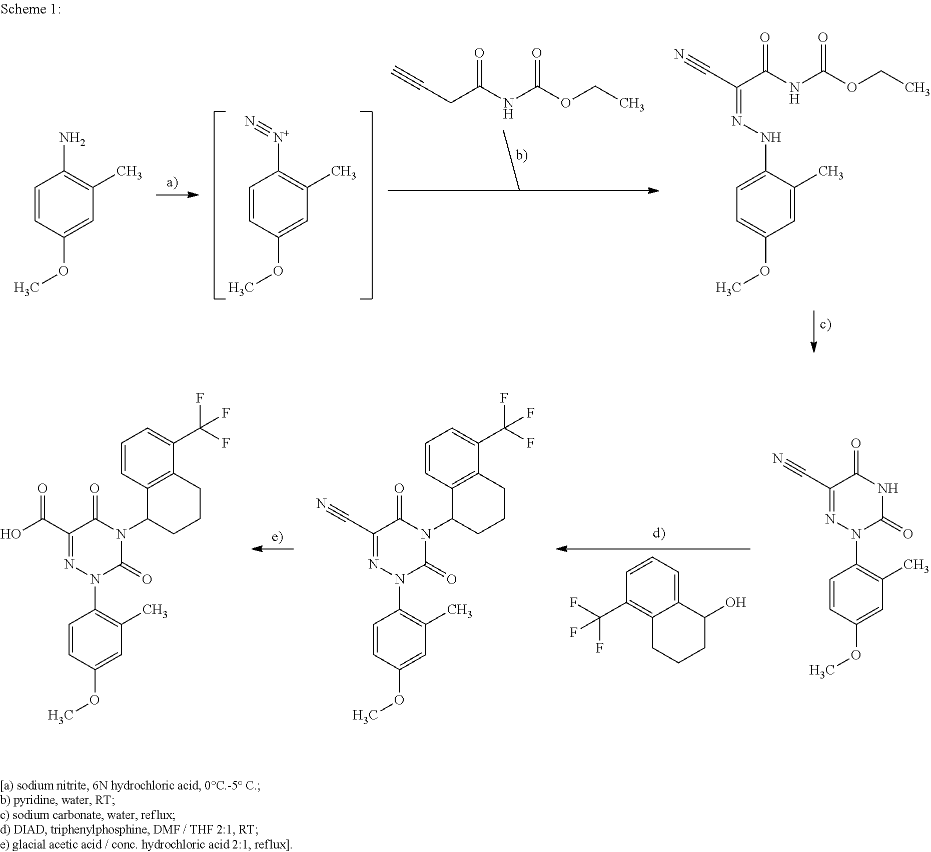

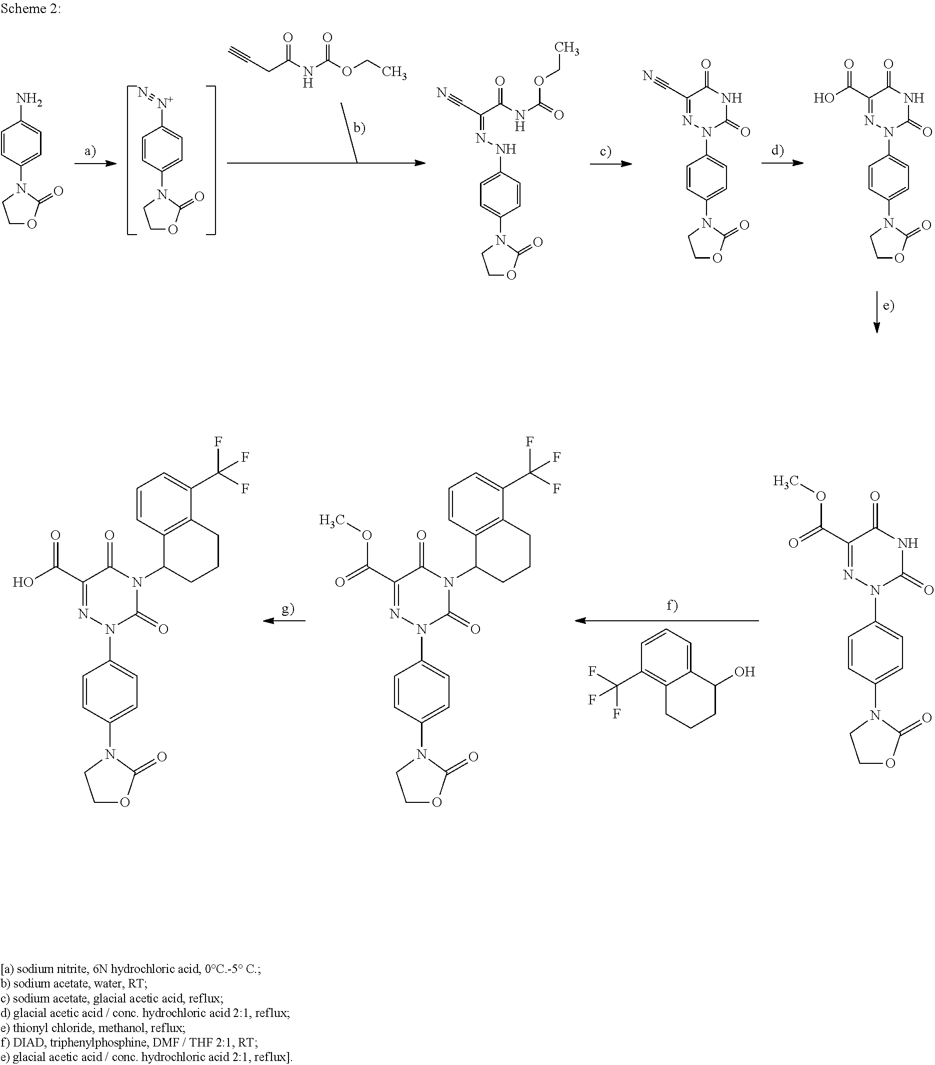

The compounds of the formulae (II), (III) and (VI) are commercially available, known from the literature or can be prepared in analogy to processes known from the literature.

Further compounds of the invention can optionally also be prepared by conversions of functional groups of individual substituents, especially those listed for R³, proceeding from the compounds of the formula (I) obtained by above processes. These conversions are performed as described in the present experimental section, by customary methods known to those skilled in the art and include, for example, reactions such as nucleophilic and electrophilic substitutions, oxidations, reductions, hydrogenations, transition metal-catalysed coupling reactions, eliminations, alkylation, amination, esterification, ester hydrolysis, etherification, ether cleavage, formation of carbonamides, and introduction and removal of temporary protecting groups,

The compounds of the invention have valuable pharmacological properties and can be used for treatment and/or prophylaxis of diseases in humans and animals.

The compounds of the invention are chymase inhibitors and are therefore suitable for treatment and/or prophylaxis of cardiovascular, inflammatory, allergic and/or fibrotic disorders.

In the context of the present invention, disorders of the cardiovascular system or cardiovascular disorders are understood to mean, for example, the following disorders: acute and chronic heart failure, arterial hypertension, coronary heart disease, stable and unstable angina pectoris, myocardial ischaemia, myocardial infarction, shock, atherosclerosis, cardiac hypertrophy, cardiac fibrosis, atrial and ventricular arrhythmias, transitory and ischaemic attacks, stroke, pre-eclampsia, inflammatory cardiovascular disorders, peripheral and cardiac vascular disorders, peripheral perfusion disorders, arterial pulmonary hypertension, spasms of the coronary arteries and peripheral arteries, thromboses, thromboembolic disorders, oedema development, for example pulmonary oedema, cerebral oedema, renal oedema or heart failure-related oedema, and restenoses such as after thrombolysis treatments, percutaneous transluminal angioplasty (PTA), transluminal coronary angioplasty (PTCA), heart transplants and bypass operations, and micro- and macrovascular damage (vasculitis), reperfusion damage, arterial and venous thromboses, microalbuminuria, myocardial insufficiency, endothelial dysfunction, elevated levels of fibrinogen and of low-density LDL and elevated concentrations of plasminogen activator/inhibitor 1 (PAI-1).

In the context of the present invention, the term “heart failure” also includes more specific or related types of disease, such as acutely decompensated heart failure, right heart failure, left heart failure, global failure, ischaemic cardiomyopathy, dilated cardiomyopathy, congenital heart defects, heart valve defects, heart failure associated with heart valve defects, mitral stenosis, mitral insufficiency, aortic stenosis, aortic insufficiency, tricuspid stenosis, tricuspid insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, and diastolic and systolic heart failure.

The compounds according to the invention are further suitable for the prophylaxis and/or treatment of polycystic kidney disease (PCKD) and of the syndrome of inappropriate ADH secretion (SIADH).

The compounds of the invention are also suitable for treatment and/or prophylaxis of kidney disorders, in particular of acute and chronic renal insufficiency and acute and chronic renal failure.

In the context of the present invention, the term “acute renal insufficiency” encompasses acute manifestations of kidney disease, of kidney failure and/or renal insufficiency with and without the need for dialysis, and also underlying or related renal disorders such as renal hypoperfusion, intradialytic hypotension, volume deficiency (e.g. dehydration, blood loss), shock, acute glomerulonephritis, haemolytic-uraemic syndrome (HUS), vascular catastrophe (arterial or venous thrombosis or embolism), cholesterol embolism, acute Bence-Jones kidney in the event of plasmacytoma, acute supravesicular or subvesicular efflux obstructions, immunological renal disorders such as kidney transplant rejection, immune complex-induced renal disorders, tubular dilatation, hyperphosphataemia and/or acute renal disorders characterized by the need for dialysis, including in the case of partial resections of the kidney, dehydration through forced diuresis, uncontrolled blood pressure rise with malignant hypertension, urinary tract obstruction and infection and amyloidosis, and systemic disorders with glomerular factors, such as rheumatological-immunological systemic disorders, for example lupus erythematodes, renal artery thrombosis, renal vein thrombosis, analgesic nephropathy and renal tubular acidosis, and x-ray contrast agent- and medicament-induced acute interstitial renal disorders.

In the context of the present invention, the term “chronic renal insufficiency” encompasses chronic manifestations of kidney disease, of kidney failure and/or renal insufficiency with and without the need for dialysis, and also underlying or related renal disorders such as renal hypoperfusion, intradialytic hypotension, obstructive uropathy, glomerulopathy, glomerular and tubular proteinuria, renal oedema, haematuria, primary, secondary and chronic glomerulonephritis, membranous and membranoproliferative glomerulonephritis, Alport syndrome, glomerulosclerosis, tubulointerstitial disorders, nephropathic disorders such as primary and congenital kidney disease, renal inflammation, immunological renal disorders such as kidney transplant rejection, immune complex-induced renal disorders, diabetic and non-diabetic nephropathy, pyelonephritis, renal cysts, nephrosclerosis, hypertensive nephrosclerosis and nephrotic syndrome, which can be characterized diagnostically, for example, by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, glomerular and arteriolar lesions, tubular dilatation, hyperphosphataemia and/or the need for dialysis, and in the event of renal cell carcinoma, after partial resections of the kidney, dehydration through forced diuresis, uncontrolled blood pressure rise with malignant hypertension, urinary tract obstruction and infection and amyloidosis, and systemic disorders with glomerular factors, such as rheumatological-immunological systemic disorders, for example lupus erythematodes, and also renal artery stenosis, renal artery thrombosis, renal vein thrombosis, analgesic nephropathy and renal tubular acidosis. In addition, x-ray contrast agent- and medicament-induced chronic interstitial renal disorders, metabolic syndrome and dyslipidaemia. The present invention also encompasses the use of the compounds of the invention for the treatment and/or prophylaxis of sequelae of renal insufficiency, for example pulmonary oedema, heart failure, uraemia, anaemia, electrolyte disorders (for example hyperkalaemia, hyponatraemia) and disorders in bone and carbohydrate metabolism.

In addition, the compounds according to the invention are also suitable for treatment and/or prophylaxis of pulmonary arterial hypertension (PAH) and other forms of pulmonary hypertension (PH), of chronic obstructive pulmonary disease (COPD), of acute respiratory distress syndrome (ARDS), of acute lung injury (ALI), of alpha-1-antitrypsin deficiency (AATD), of pulmonary fibrosis, of pulmonary emphysema (for example pulmonary emphysema caused by cigarette smoke), of cystic fibrosis (CF), of acute coronary syndrome (ACS), heart muscle inflammation (myocarditis) and other autoimmune cardiac disorders (pericarditis, endocarditis, valvolitis, aortitis, cardiomyopathy), cardiogenic shock, aneurysms, sepsis (SIRS), multiple organ failure (MODS, MOF), inflammation disorders of the kidney, chronic intestinal disorders (IBD, Crohn's Disease, UC), pancreatitis, peritonitis, rheumatoid disorders, inflammatory skin disorders and inflammatory eye disorders.

The compounds according to the invention can furthermore be used for treatment and/or prophylaxis of asthmatic disorders of varying severity with intermittent or persistent characteristics (refractive asthma, bronchial asthma, allergic asthma, intrinsic asthma, extrinsic asthma, medicament- or dust-induced asthma), of various forms of bronchitis (chronic bronchitis, infectious bronchitis, eosinophilic bronchitis), of Bronchiolitis obliterans, bronchiectasis, pneumonia, idiopathic interstitial pneumonia, farmer's lung and related disorders, of coughs and colds (chronic inflammatory cough, iatrogenic cough), inflammation of the nasal mucosa (including medicament-related rhinitis, vasomotoric rhinitis and seasonal allergic rhinitis, for example hay fever) and of polyps.

The compounds according to the invention are also suitable for treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term “fibrotic disorders” encompasses particularly the following terms: hepatic fibrosis, cirrhosis of the liver, pulmonary fibrosis, endomyocardial fibrosis, cardiomyopathy, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy and proliferative vitroretinopathy.

The compounds according to the invention are also suitable for controlling postoperative scarring, for example as a result of glaucoma operations.

Furthermore, the compounds according to the invention can also be used cosmetically for ageing and keratinized skin.

In addition, the compounds of the invention can also be used for treatment and/or prophylaxis of dyslipidaemias (hypercholesterolaemia, hypertriglyceridaemia, elevated concentrations of the postprandial plasma triglycerides, hypoalphalipoproteinaemia, combined hyperlipidaemias), nephropathy and neuropathy), cancers (skin cancer, brain tumours, breast cancer, bone marrow tumours, leukaemias, liposarcomas, carcinoma of the gastrointestinal tract, of the liver, pancreas, lung, kidney, urinary tract, prostate and genital tract, and also malignant tumours in the lymphoproliferative system, for example Hodgkin's and non-Hodgkin's lymphoma), of disorders of the gastrointestinal tract and of the abdomen (glossitis, gingivitis, periodontitis, oesophagitis, eosinophilic gastroenteritis, mastocytosis, Crohn's disease, colitis, proctitis, pruritus ani, diarrhoea, coeliac disease, hepatitis, chronic hepatitis, hepatic fibrosis, cirrhosis of the liver, pancreatitis and cholecystitis), skin disorders (allergic skin disorders, psoriasis, acne, eczema, neurodermitis, various forms of dermatitis, and also keratitis, bullosis, vasculitis, cellulitis, panniculitis, lupus erythematodes, erythema, lymphoma, skin cancer, Sweet's syndrome, Weber-Christian syndrome, scarring, warts, chillblains), of disorders of the skeletal bone and of the joints, and also of the skeletal muscle (various forms of arthritis, various forms of arthropathies, scleroderma and of further disorders with an inflammatory or immunological component, for example paraneoplastic syndrome, in the event of rejection reactions after organ transplants and for wound healing and angiogenesis, especially in the case of chronic wounds.

The compounds of the formula (I) according to the invention are additionally suitable for treatment and/or prophylaxis of ophthalmologic disorders, for example glaucoma, normotensive glaucoma, high intraocular pressure and combinations thereof, of age-related macular degeneration (AMD), of dry or non-exudative AMD, moist or exudative or neovascular AMD, choroidal neovascularization (CNV), detached retina, diabetic retinopathy, atrophic lesions to the retinal pigment epithelium (RPE), hypertrophic lesions to the retinal pigment epithelium (RPE), diabetic macular oedema, retinal vein occlusion, choroidal retinal vein occlusion, macular oedema, macular oedema due to retinal vein occlusion, angiogenesis at the front of the eye, for example corneal angiogenesis, for example following keratitis, cornea transplant or keratoplasty, corneal angiogenesis due to hypoxia (extensive wearing of contact lenses), pterygium conjunctiva, subretinal oedema and intraretinal oedema.

In addition, the compounds of the formula (I) according to the invention are suitable for the treatment and/or prophylaxis of elevated and high intraocular pressure resulting from traumatic hyphaema, periorbital oedema, postoperative viscoelastic retention, intraocular inflammation, use of corticosteroids, pupillary block or idiopathic causes, and of elevated intraocular pressure following trabeculectomy and due to pre-operative conditions.

The present invention further provides for the use of the compounds according to the invention for treatment and/or prophylaxis of disorders, especially the disorders mentioned above.

The present invention further provides for the use of the compounds according to the invention for production of a medicament for treatment and/or prophylaxis of disorders, especially the disorders mentioned above.

The present invention further provides the compounds according to the invention for use in a method for treatment and/or prophylaxis of heart failure, pulmonary hypertension, chronic obstructive pulmonary disease, asthma, kidney failure, nephropathy, fibrotic disorders of the internal organs and dermatological fibroses.

The compounds of the invention can be used alone or, if required, in combination with other active ingredients. Accordingly, the present invention further provides medicaments comprising at least one of the compounds according to the invention and one or more further active compounds, especially for treatment and/or prophylaxis of the aforementioned disorders. Preferred examples of active compounds suitable for combinations include:

compounds which inhibit the signal transduction cascade, by way of example and with preference from the group of the kinase inhibitors, especially from the group of the tyrosine kinase and/or serine/threonine kinase inhibitors;

compounds which inhibit the degradation and alteration of the extracellular matrix, by way of example and with preference inhibitors of the matrix metalloproteases (MMPs), especially inhibitors of stromelysin, collagenases, gelatinases and aggrecanases (in this context particularly of MMP-1, MMP-3, MMP-8, MMP-9, MMP-10, MMP-11 and MMP-13) and of metalloelastase (MMP-12);

compounds which block the binding of serotonin to its receptors, by way of example and with preference antagonists of the 5-HT_{2b} receptor;

organic nitrates and NO donors, for example sodium nitroprusside, nitroglycerin, isosorbide mononitrate, isosorbide dinitrate, molsidomine or SIN-1, and inhaled NO;

NO-independent but haem-dependent stimulators of soluble guanylate cyclase, such as especially the compounds described in WO 00/06568, WO 00/06569, WO 02/42301 and WO 03/095451;

NO- and haem-independent activators of soluble guanylate cyclase, such as especially the compounds described in WO 01/19355, WO 01/19776, WO 01/19778, WO 01/19780, WO 02/070462 and WO 02/070510;

prostacyclin analogues, by way of example and with preference iloprost, beraprost, treprostinil or epoprostenol;

compounds which inhibit soluble epoxide hydrolase (sEH), for example N,N′-dicyclohexylurea, 12-(3-adamantan-1-ylureido)dodecanoic acid or 1-adamantan-1-yl-3-{5-[2-(2-ethoxyethoxy)ethoxy]pentyl}urea;

compounds which influence the energy metabolism of the heart, by way of example and with preference etomoxir, dichloroacetate, ranolazine or trimetazidine;

compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil and tadalafil;

antithrombotic agents, by way of example and with preference from the group of the platelet aggregation inhibitors, the anticoagulants or the profibrinolytic substances;

hypotensive active ingredients, for example and with preference from the group of calcium antagonists, angiotensin All antagonists, ACE inhibitors, vasopeptidase inhibitors, endothelin antagonists, renin inhibitors, alpha-receptor blockers, beta-receptor blockers, mineralocorticoid receptor antagonists, and rho kinase inhibitors and the diuretics;

vasopressin receptor antagonists, for example and with preference conivaptan, tolvaptan, lixivaptan, mozavaptan, satavaptan, SR-121463, RWJ 676070 or BAY 86-8050;

bronchodilatory agents, by way of example and with preference from the group of the beta-adrenergic receptor agonists, such as especially albuterol, isoproterenol, metaproterenol, terbutalin, formoterol or salmeterol, or from the group of the anticholinergics, such as especially ipratropium bromide;

anti-inflammatory agents, by way of example and with preference from the group of the glucocorticoids, such as especially prednisone, prednisolone, methylprednisolone, triamcinolone, dexamethasone, beclomethasone, betamethasone, flunisolide, budesonide or fluticasone; and/or

active compounds altering lipid metabolism, for example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

In a preferred embodiment of the invention, the compounds according to the invention are used in combination with a kinase inhibitor, by way of example and with preference bortezomib, canertinib, erlotinib, gefitinib, imatinib, lapatinib, lestaurtinib, lonafarnib, pegaptinib, pelitinib, semaxanib, sorafenib, regorafenib, sunitinib, tandutinib, tipifarnib, vatalanib, fasudil, lonidamine, leflunomide, BMS-3354825 or Y-27632.

In a preferred embodiment of the invention, the compounds according to the invention are used in combination with a serotonin receptor antagonist, by way of example and with preference PRX-08066.

Antithrombotic agents are preferably understood to mean compounds from the group of the platelet aggregation inhibitors, the anticoagulants or the profibrinolytic substances.

In a preferred embodiment of the invention, the compounds of the invention are administered in combination with a platelet aggregation inhibitor, by way of example and with preference aspirin, clopidogrel, ticlopidine or dipyridamole.

In a preferred embodiment of the invention, the compounds according to the invention are administered in combination with a thrombin inhibitor, by way of example and with preference ximelagatran, melagatran, bivalirudin or clexane.

In a preferred embodiment of the invention, the compounds of the invention are administered in combination with a GPIIb/IIIa antagonist, by way of example and with preference tirofiban or abciximab.

In a preferred embodiment of the invention, the compounds according to the invention are administered in combination with a factor Xa inhibitor, by way of example and with preference rivaroxaban, DU-176b, fidexaban, razaxaban, fondaparinux, idraparinux, PMD-3112, YM-150, KFA-1982, EMD-503982, MCM-17, mLN-1021, DX 9065a, DPC 906, JTV 803, SSR-126512 or SSR-128428.

In a preferred embodiment of the invention, the compounds of the invention are administered in combination with heparin or with a low molecular weight (LMW) heparin derivative.

In a preferred embodiment of the invention, the compounds of the invention are administered in combination with a vitamin K antagonist, by way of example and with preference coumarin.

Hypotensive agents are preferably understood to mean compounds from the group of calcium antagonists, angiotensin All antagonists, ACE inhibitors, endothelin antagonists, renin inhibitors, alpha-receptor blockers, beta-receptor blockers, mineralocorticoid receptor antagonists, rho kinase inhibitors, and the diuretics.

In a preferred embodiment of the invention, the compounds of the invention are administered in combination with a calcium antagonist, by way of example and with preference nifedipine, amlodipine, verapamil or diltiazem.

In a preferred embodiment of the invention, the compounds of the invention are administered in combination with an alpha-1-receptor blocker, by way of example and with preference prazosin.

In a preferred embodiment of the invention, the compounds of the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin All antagonist, by way of example and with preference losartan, candesartan, valsartan, telmisartan or embursatan.

In a preferred embodiment of the invention, the compounds of the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds of the invention are administered in combination with an endothelin antagonist, by way of example and with preference bosentan, darusentan, ambrisentan or sitaxsentan.

In a preferred embodiment of the invention, the compounds of the invention are administered in combination with a renin inhibitor, by way of example and with preference aliskiren, SPP-600 or SPP-800.

In a preferred embodiment of the invention, the compounds of the invention are administered in combination with a mineralocorticoid receptor antagonist, by way of example and with preference spironolactone or eplerenone.

In a preferred embodiment of the invention, the compounds according to the invention are administered in combination with a rho kinase inhibitor, by way of example and with preference fasudil, Y-27632, SLx-2119, BF-66851, BF-66852, BF-66853, KI-23095, SB-772077, GSK-269962A or BA-1049.

In a preferred embodiment of the invention, the compounds according to the invention are administered in combination with a diuretic, by way of example and with preference furosemide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the compounds according to the invention are administered in combination with a CETP inhibitor, by way of example and with preference torcetrapib (CP-529 414), JJT-705 or CETP vaccine (Avant).

In a preferred embodiment of the invention, the compounds of the invention are administered in combination with a thyroid receptor agonist such as, for example and preferably, D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494 or TAK-475.

In a preferred embodiment of the invention, the compounds of the invention are administered in combination with an ACAT inhibitor, by way of example and with preference avasimibe, melinamide, pactimibe, eflucimibe or SMP-797.

In a preferred embodiment of the invention, the compounds of the invention are administered in combination with an MTP inhibitor, by way of example and with preference implitapide, BMS-201038, R-103757 or JTT-130.

In a preferred embodiment of the invention, the compounds of the invention are administered in combination with a PPAR-gamma agonist, by way of example and with preference pioglitazone or rosiglitazone.

In a preferred embodiment of the invention, the compounds of the invention are administered in combination with a PP AR-delta agonist, by way of example and with preference GW 501516 or BAY 68-5042.

In a preferred embodiment of the invention, the compounds of the invention are administered in combination with a cholesterol absorption inhibitor, by way of example and with preference ezetimibe, tiqueside or pamaqueside.

In a preferred embodiment of the invention, the compounds of the invention are administered in combination with a lipase inhibitor, by way of example and with preference orlistat.

In a preferred embodiment of the invention, the compounds of the invention are administered in combination with a polymeric bile acid adsorbent, by way of example and with preference cholestyramine, colestipol, colesolvam, CholestaGel or colestimide.

In a preferred embodiment of the invention, the compounds of the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds of the invention are administered in combination with a lipoprotein(a) antagonist, by way of example and with preference gemcabene calcium (CI-1027) or nicotinic acid.

The present invention further provides medicaments which comprise at least one compound of the invention, typically together with one or more inert, nontoxic, pharmaceutically suitable excipients, and for the use thereof for the aforementioned purposes.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which work according to the prior art and release the compounds of the invention rapidly and/or in a modified manner and which contain the compounds of the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can bypass an absorption step (e.g. intravenously, intraarterially, intracardially, intraspinally or intralumbally) or include an absorption (e.g. inhalatively, intramuscularly, subcutaneously, intracutaneously, percutaneously or intraperitoneally).

Administration forms suitable for parenteral administration include preparations for injection and infusion in the form of solutions, suspensions, emulsions, lyophilizates or sterile powders.

For the other administration routes, suitable examples are inhalation medicaments (including powder inhalers, nebulizers, aerosols), nasal drops, solutions or sprays; tablets for lingual, sublingual or buccal administration, films/oblates or capsules, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Oral and parenteral administration are preferred, especially oral, intravenous and inhalative administration.

The compounds of the invention can be converted to the administration forms mentioned. This can be done in a manner known per se, by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration the dosage is about 0.01 to 100 mg/kg, preferably about 0.01 to 20 mg/kg and most preferably 0.1 to 10 mg/kg of body weight.

It may nevertheless be necessary in some cases to deviate from the stated amounts, specifically as a function of the body weight, route of administration, individual response to the active ingredient, nature of the preparation and time or interval over which administration takes place. Thus, in some cases less than the abovementioned minimum amount may be sufficient, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight. Solvent ratios, dilution ratios and concentration data for liquid/liquid solutions, unless indicated otherwise, are based in each case on volume.

A. EXAMPLES

Abbreviations:
Ac acetyl
aq. aqueous, aqueous solution
br.d broad doublet (NMR)
br.m broad multiplet (NMR)
br.s broad singlet (NMR)
br.t broad triplet (NMR)
Ex. Example
c concentration
cat. catalytic
TLC thin-layer chromatography
DCI direct chemical ionization (in MS)
DIAD diisopropyl azodicarboxylate
DIEA N,N-diisopropylethylamine
DMAP 4-N,N-dimethylaminopyridine
DMF dimethylformamide
DMSO dimethyl sulfoxide
EDC N′-(3-dimethylaminopropyl)-N-ethylcarbodiimide hydrochloride
ee enantiomeric excess
eq. equivalent(s)
ESI electrospray ionization (in MS)
Et ethyl
GC-MS gas chromatography-coupled mass spectrometry
h hour(s)
HATU O-(7-azabenzotriazol-1-yl)-N,N,N′,N′-tetramethyluronium hexafluorophosphate
HOBt 1-hydroxy-1H-benzotriazole hydrate
HPLC high-pressure, high-performance liquid chromatography
conc. concentrated
LC-MS liquid chromatography-coupled mass spectrometry
Me methyl
min minute(s)
MS mass spectrometry
MTBE methyl tent-butyl ether
NMR nuclear magnetic resonance spectrometry
Pd/C palladium on activated carbon
Ph phenyl
PyBOP benzotriazol-1-yloxytris(pyrrolidino)phosphonium hexafluorophosphate
quant. quantitative (in yield)
rac racemic, racemate
RT room temperature
Rₜ retention time (in HPLC)
tBu tert-butyl
tert tertiary
TFA trifluoroacetic acid
TFAA trifluoroacetic anhydride
THF tetrahydrofuran
TPPO triphenylphosphine oxide
UV ultraviolet spectrometry
v/v volume to volume ratio (of a solution)

HPLC, GC-MS and LC-MS Methods:

Method 1 (LC-MS): Instrument: Waters ACQUITY SQD UPLC system; column: Waters Acquity UPLC HSS T3 1.8μ 50×1 mm; mobile phase A: 1 l of water+0.25 ml of 99% strength formic acid, mobile phase B: 1 l of acetonitrile+0.25 ml of 99% strength formic acid; gradient 0.0 min 90% A→1.2 min 5% A→2.0 min 5% A; oven: 50° C.; flow rate: 0.40 ml/min; UV detection: 210-400 nm.

Method:2 (LC-MS): MS instrument type: Micromass ZQ; HPLC instrument type: HP 1100 Series; UV DAD; column: Phenomenex Gemini 3μ, 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate 0.0 min 1 ml/min, 2.5 min/3.0 min/4.5 min 2 ml/min; oven: 50° C.; UV detection: 210 nm.

Method 3 (LC-MS): Instrument: Agilent MS Quad 6150; HPLC: Agilent 1290; column: Waters Acquity UPLC HSS T3 1.8μ 50×2.1 mm; mobile phase A: 1l of water+0.25 ml of 99% strength formic acid, mobile phase B: 1 l of acetonitrile+0.25 ml of 99% strength formic acid; gradient: 0.0 min 90% A→0.3 min 90% A→1.7 min 5% A→3.0 min 5% A oven: 50° C.; flow rate: 1.20 ml/min; UV detection: 205-305 nm.

Method 4 (preparative HPLC): Column: Reprosil C_{18, 10} μm, 250 mm×30 mm Mobile phase A: formic acid 0.1% in water, mobile phase B: acetonitrile; flow rate: 50 ml/min; programme: 0 to 6 min: 90% A/10% B; 6 min to 27 min: gradient to 95% B; 27 min to 38 min 95% B; 38 min to 39 min gradient to 10% B; 39 min to 43 min (end): 60% A/40% B. Slight variations in the gradient are possible.

Method 5 (preparative HPLC): As Method 4, but using the Chromatorex C_{18 5 μ}m, 250×20 mm column.

Method 6 (LC-MS): Instrument: Micromass Quattro Premier with Waters UPLC Acquity; column^{.}

Thermo Hypersil GOLD 1.9μ 50×1 mm; mobile phase A: 1 l of water+0.5 ml of 50% strength formic acid, mobile phase B: 1 l of acetonitrile+0.5 ml of 50% strength formic acid; gradient: 0.0 min 97% A→0.5 min 97% A→3.2 min 5% A→4.0 min 5% A oven: 50° C.; flow rate: 0.3 ml/min; UV detection: 210 nm.

Method 7 (MS; ESI): Instrument: Waters ZQ 2000; electrospray ionization; mobile phase A: 1 l of water+0.25 ml of 99% strength formic acid, mobile phase B: 1 l of acetonitrile+0.25 ml of 99% strength formic acid; 25% A, 75% B; flow rate: 0.25 ml/min.

Starting Materials and Intermediates:

### Example 1A

Ethyl {2-cyano-2-[2-(4-methoxy-2-methylphenyl)hydrazinylidene]acetyl}carbamate

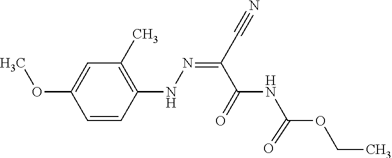 [SMILES: CCOC(=O)NC(=O)/C(C#N)=N\Nc1ccc(OC)cc1C]

A solution of 5.00 g (36.45 mmol) of 4-methoxy-2-methylaniline in 50 ml of 6N aqueous hydrochloric acid was cooled to 0° C. A solution of 2.51 g (36.45 mmol) of sodium nitrite in 15 ml of water was added dropwise such that the reaction temperature did not exceed 5° C. The mixture was then stirred at 0° C. for a further 30 min In another flask, 6.09 g (39.0 mmol) of ethyl (cyanoacetyl)carbamate were dissolved in 150 ml of water, 30 ml of pyridine were added and the mixture was cooled to 0° C. The solution prepared beforehand of the diazonium salt of 4-methoxy-2-methylaniline was slowly added dropwise with stirring, and the reaction mixture was then stirred at RT for 30 min. The solid formed was filtered off with suction, washed with water and dried under HV. This gave 7.42 g (purity 64%) of the title compound.

LC-MS (Method 2): Rₜ=2.05 min., m/z=305 (M+H)⁺

### Example 2A

2-(4-Methoxy-2-methylphenyl)-3,5-dioxo-2,3,4,5-tetrahydro-1,2,4-triazine-6-carbonitrile

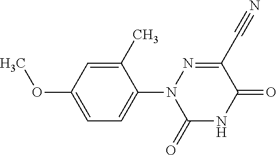 [SMILES: COc1ccc(-n2nc(C#N)c(=O)[nH]c2=O)c(C)c1]

2.91 g (27.5 mmol) of sodium carbonate were added to a suspension of 7.4 g of the crude product from Example 1A in 60 ml of water, and the mixture was heated at 100° C. for 2.5 h. After cooling to RT, the pH was adjusted to pH=1 by addition of 1N aqueous hydrochloric acid. The solid formed was filtered off with suction, washed with petroleum ether and dried under HV. This gave 4.46 g (45% of theory over two steps) of the title compound.

¹H-NMR (400 MHz, DMSO-d₆): δ[ppm]=2.15 (s, 3H), 3.79 (s, 3H), 6.84-6.95 (m, 2H), 7.27 (d, 1H), 12.94 (br.s, 1H).

### Example 3A

3,5-Dioxo-2-[4-(2-oxo-1,3-oxazolidin-3-yl)phenyl]-2,3,4,5-tetrahydro-1,2,4-triazine-6-carbonitrile

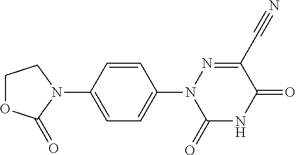 [SMILES: N#Cc1nn(-c2ccc(N3CCOC3=O)cc2)c(=O)[nH]c1=O]

Preparation of solution 1: A solution of 1.49 g (9.54 mmol) of ethyl (cyanoacetyl)carbamate in 5 ml of ethanol was added to a solution of 3.44 g (42 0 mmol) of sodium acetate in 13 ml of water, and the mixture was stirred at RT for 2 h.

Preparation of solution 2: 5 ml of ethanol, 8 ml of water and 1.2 ml of conc. hydrochloric acid were added successively to 1.70 g (9.54 mmol) of 3-(4-aminophenyl)-1,3-oxazolidin-2-one (for preparation: see WO2010/019903, p.222, Method 38; or Farmaco Sci. Ed. (1969), 179). The resulting mixture was cooled to 0° C., and a solution of 658 g (9.54 mmol) of sodium nitrite in 5 ml of water was slowly added such that the reaction temperature did not exceed 2° C. The resulting solution was stirred at 0° C. for another 30 min.

The cold solution 2 was stirred into solution 1 and stirring of the mixture was continued at RT overnight, resulting in the precipitation of a solid. 40 ml of 6N aqueous hydrochloric acid were added, the suspension was stirred for a further 30 min and the solid was filtered off with suction.

The solid was washed with 25 ml of water, stirred with 50 ml of 2-propanol and filtered off again. The solid was then suspended in 80 ml of glacial acetic acid. 1.15 g (14.0 mmol) of sodium acetate were added to this suspension. The mixture was heated at reflux temperature overnight. After cooling to RT, the resulting solution was poured into 1 l of ice-water and the mixture was stirred for 10 min The product formed was filtered off with suction and dried under HV. This gave 1.57 g (55% of theory) of the title compound.

LC-MS (Method 1): Rₜ=0.70 min., m/z=300 (M+H)⁺

¹H-NMR (400 MHz, DMSO-d₆): δ[ppm]=4.10 (t, 2H), 4.47 (t, 2H), 7.50 (d, 2H), 7.70 (d, 2H), 13.02 (br. s, 1H).

### Example 4A

3,5-Dioxo-2-[4-(2-oxoimidazolidin-1-yl)phenyl]-2,3,4,5-tetrahydro-1,2,4-triazine-6-carbonitrile

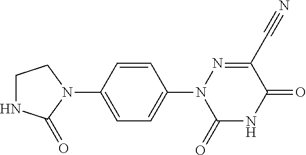 [SMILES: N#Cc1nn(-c2ccc(N3CCNC3=O)cc2)c(=O)[nH]c1=O]

Analogously to Example 3A, the title compound was prepared from 500 mg (2.82 mmol) of 1-(4-aminophenyl)imidazolidin-2-one (preparation see: P. Stabile et al., Tetrahedron Letters 2010, 51 (24), 3232-3235) and 441 mg (2.82 mmol) of ethyl (cyanoacetyl)carbamate, with the difference that the solution of the crude product in glacial acetic acid was separated completely by preparative HPLC (Method 4). This gave 173 mg (16% of theory, purity 80%) of the title compound.

LC-MS (Method 1): Rₜ=0.58 min., m/z=299 (M+H)⁺

¹H-NMR (400 MHz, DMSO-d₆): δ[ppm]=3.38-3.49 (m, 2H), 3.88 (dd, 2H), 7.37-7.43 (m, 2H), 7.65-7.70 (m, 2H), 12.98 (br. s., 1H).

### Example 5A

2-(3-Methyl-2-oxo-2,3-dihydro-1,3-benzoxazol-6-yl)-3,5-dioxo-2,3,4,5-tetrahydro-1,2,4-triazine-6-carbonitrile

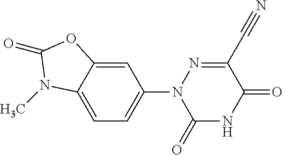 [SMILES: Cn1c(=O)oc2cc(-n3nc(C#N)c(=O)[nH]c3=O)ccc21]

The title compound was prepared and isolated analogously to Example 3A from 1.53 g (9.30 mmol) of 6-amino-3-methyl-1,3-benzoxazol-2(3H)-one and 1.45 g (9.30 mmol) of ethyl (cyanoacetyl)carbamate. This gave 0.82 g (30% of theory) of the title compound.

LC-MS (Method 1): Rₜ=0.71 min., m/z=286 (M+H)⁺

¹H-NMR (400 MHz, DMSO-d₆): δ[ppm]=3.38 (s, 3H), 7.32-7.42 (m, 2H), 7.48 (d, 1H), 13.07 (br. s, 1H).

### Example 6A

3,5-Dioxo-2-[4-(2-oxo-1,3-oxazolidin-3-yl)phenyl]-2,3,4,5-tetrahydro-1,2,4-triazine-6-carboxylic acid

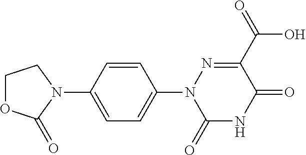 [SMILES: O=C(O)c1nn(-c2ccc(N3CCOC3=O)cc2)c(=O)[nH]c1=O]

13.8 ml of glacial acetic acid and 6.9 ml of conc. hydrochloric acid were added to 1.50 g (5.01 mmol) of the compound from Example 3A, and the mixture was heated at reflux temperature for 2.5 days. After cooling to RT, 200 ml of ice-cooled water were added to the solution and the mixture was extracted with ethyl acetate. The organic phase was dried over sodium sulfate. The solvent was removed on a rotary evaporator and the residue was dried under HV. This gave 1.20 g of the title compound (purity about 42% according to LC-MS). The aqueous phase was also concentrated to dryness on a rotary evaporator. The residue (380 mg) contained about 52% of the title compound (LC-MS). Both residues were combined and converted into the corresponding methyl ester (see Example 7A).

LC-MS (Method 1): Rₜ=0.23 min., m/z=319 (M+H)⁺

### Example 7A

Methyl 3,5-dioxo-2-[4-(2-oxo-1,3-oxazolidin-3-yl)phenyl]-2,3,4,5-tetrahydro-1,2,4-triazine-6-carboxylate

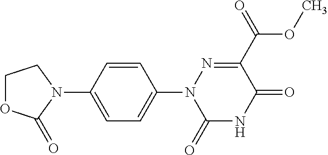 [SMILES: COC(=O)c1nn(-c2ccc(N3CCOC3=O)cc2)c(=O)[nH]c1=O]

The combined residues from Example 6A (1.58 g) were taken up in 100 ml of methanol, and 1.81 ml of thionyl chloride were added dropwise to the suspension. The reaction mixture was then heated at reflux overnight. After cooling to RT, 100 ml of diethyl ether were added. The solid formed was filtered off with suction and dried under HV. This gave 418 mg (25% of theory over two steps) of the title compound.

LC-MS (Method 1): Rₜ=0.58 min., m/z=333 (M+H)⁺

¹H-NMR (400 MHz, DMSO-d₆): δ[ppm]=3.81 (s, 3H), 4.06-4.14 (m, 2H), 4.41-4.51 (m, 2H), 7.51 (d, 2H), 7.68 (d, 2H), 12.55 (s, 1H).

### Example 8A

2-(4-Methoxyphenyl)-3,5-dioxo-4-[5-(trifluoromethyl)-1,2,3,4-tetrahydronaphthalen-1-yl]-2,3,4,5-tetrahydro-1,2,4-triazine-6-carbonitrile (racemate)

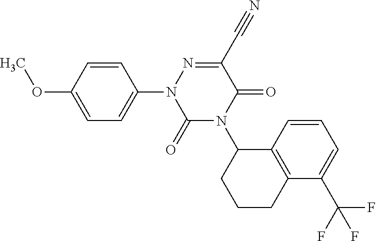 [SMILES: COc1ccc(-n2nc(C#N)c(=O)n(C3CCCc4c3cccc4C(F)(F)F)c2=O)cc1]

50.0 mg (0.205 mmol) of 2-(4-methoxyphenyl)-3,5-dioxo-2,3,4,5-tetrahydro-1,2,4-triazine-6-carbonitrile (preparation: see J. Slouka, Monatshefte für Chemie 1968, 99 (5), 1808), 53.1 mg (0.25 mmol) of 5-(trifluoromethyl)-1,2,3,4-tetrahydronaphthalen-1-ol (racemate) and 91.3 mg (0.35 mmol) of triphenylphosphine were initially charged in 1.22 ml of DMF and 0.61 ml of THF. At RT, 65 μl (0.33 mmol) of DIAD were added dropwise to this mixture, and the resulting mixture was stirred at RT for 1 h. 1 ml of IN aqueous hydrochloric acid was added with ice cooling. The mixture was stirred for another 10 min and then separated directly by preparative HPLC (Method 5). This gave 15 mg (17% of theory) of the title compound and 17 mg of a further fraction with a purity of about 60%.

LC-MS (Method 3): Rₜ=1.54 min., ESI-neg. m/z=487 (M+HCOOH—H)⁻

¹H-NMR (400 MHz, CDCl₃): δ[ppm]=1.71-1.89 (m, 1H), 2.11-2.24 (m, 2H), 2.29-2.44 (m, 1H), 2.86-3.02 (m, 1H), 3.05-3.17 (m, 1H), 3.84 (s, 3H), 6.14-6.30 (m, 1H), 6.88-7.02 (m, 2H), 7.13 (d, 1H), 7.20-7.25 (m, 1H, partially obscured by the CHCl₃ signal) 7.34 (d, 2H) 7.53 (d, 1H).

### Example 9A

4-(5-Chloro-1,2,3,4-tetrahydronaphthalen-1-yl)-2-(4-methoxyphenyl)-3,5-dioxo-2,3,4,5-tetrahydro-1,2,4-triazine-6-carbonitrile (racemate)

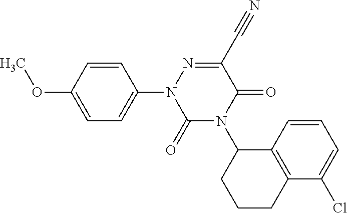 [SMILES: COc1ccc(-n2nc(C#N)c(=O)n(C3CCCc4c(Cl)cccc43)c2=O)cc1]

Analogously to Example 8A, 50.0 mg (0.205 mmol) of 2-(4-methoxyphenyl)-3,5-dioxo-2,3,4,5-tetrahydro-1,2,4-triazine-6-carbonitrile (preparation: see J. Slouka, Monatshefte für Chemie 1968, 99 (5), 1808) were reacted with 44.9 mg (0.25 mmol) of 5-chloro-1,2,3,4-tetrahydronaphthalen-1-ol (racemate). This gave 24 mg (28% of theory) of the title compound.

¹H-NMR (400 MHz, CDCl₃): δ[ppm]=1.70-1.88 (m, 1H), 2.07-2.24 (m, 2H), 2.31-2.44 (m, 1H), 2.68-2.83 (m, 1H), 3.05 (br. d, 1H), 3.84 (s, 3H), 6.12-6.24 (m, 1H), 6.85 (d, 1H), 6.96 (d, 2H), 7.07 (t, 1H), 7.23-7.27 (m, 1H, partially under the CHCl₃ signal), 7.34 (d, 2H).

### Example 10A

2-(4-Methoxyphenyl)-3,5-dioxo-4-[(1R)-4-(trifluoromethyl)-2,3-dihydro-1H-inden-1-yl]-2,3,4,5-tetrahydro-1,2,4-triazine-6-carbonitrile (R enantiomer)

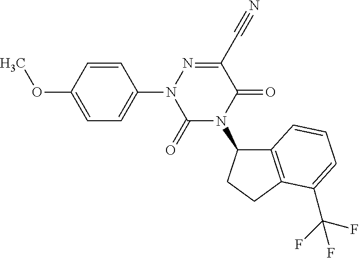 [SMILES: COc1ccc(-n2nc(C#N)c(=O)n([C@@H]3CCc4c3cccc4C(F)(F)F)c2=O)cc1]

Analogously to Example 8A, 50.0 mg (0.205 mmol) of 2-(4-methoxyphenyl)-3,5-dioxo-2,3,4,5-tetrahydro-1,2,4-triazine-6-carbonitrile (preparation: see J. Slouka, Monatshefte für Chemie 1968, 99 (5), 1808) were reacted with 49.7 mg (0.25 mmol) of (1S)-4-(trifluoromethyl)indan-1-ol (S enantiomer). This gave 17 mg (18% of theory) of the title compound.

LC-MS (Method 1): Rₜ=1.22 min., ES neg. m/z=473 (M+HCOOH—H)⁻

¹H-NMR (400 MHz, CDCl₃): δ[ppm]=2.38-2.50 (m, 1H), 2.63-2.72 (m, 1H), 3.11-3.27 (m, 1H), 3.51-3.64 (m, 1H), 3.84 (s, 3H), 6.48-6.59 (m, 1H), 6.97 (d, 2H), 7.29-7.38 (m, 4H), 7.50-7.59 (m, 1H).

### Example 11A

2-(4-Methoxy-2-methylphenyl)-3,5-dioxo-4-[5-(trifluoromethyl)-1,2,3,4-tetrahydronaphthalen-1-yl]-2,3,4,5-tetrahydro-1,2,4-triazine-6-carbonitrile (racemate)

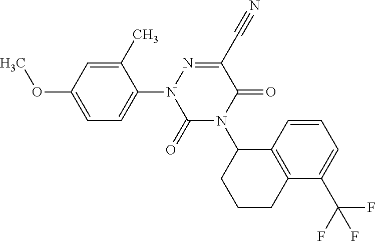 [SMILES: COc1ccc(-n2nc(C#N)c(=O)n(C3CCCc4c3cccc4C(F)(F)F)c2=O)c(C)c1]

Analogously to Example 8A, 50.0 mg (0.194 mmol) of 2-(4-methoxy-2-methylphenyl)-3,5-dioxo-2,3,4,5-tetrahydro-1,2,4-triazine-6-carbonitrile from Example 2A were reacted with 50.2 mg (0.23 mmol) of 5-(trifluoromethyl)-1,2,3,4-tetrahydronaphthalen-1-ol (racemate). This gave 20 mg (23% of theory) of the title compound.

LC-MS (Method 1): Rₜ=1.29 min., ES neg. m/z=501 (M+HCOOH—H)⁻

¹H-NMR (400 MHz, CDCl₃): δ[ppm]=1.71-1.87 (m, 1H), 2.07 (br. s, 3H), 2.13-2.25 (m, 2H), 2.28-2.42 (m, 1H), 2.86-2.98 (m, 1H), 3.05-3.15 (m, 1H), 3.81 (s, 3H), 6.15-6.28 (m, 1H), 6.75-6.86 (m, 2H), 7.12 (dd, 2H), 7.19-7.25 (m, 1H), 7.47-7.58 (m, 1H).

### Example 12A

4-(5-Chloro-1,2,3,4-tetrahydronaphthalen-1-yl)-2-(4-methoxy-2-methylphenyl)-3,5-dioxo-2,3,4,5-tetrahydro-1,2,4-triazine-6-carbonitrile (racemate)

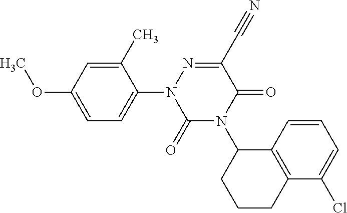 [SMILES: COc1ccc(-n2nc(C#N)c(=O)n(C3CCCc4c(Cl)cccc43)c2=O)c(C)c1]

Analogously to Example 8A, 50.0 mg (0.194 mmol) of 2-(4-methoxy-2-methylphenyl)-3,5-dioxo-2,3,4,5-tetrahydro-1,2,4-triazine-6-carbonitrile from Example 2A were reacted with 42.4 mg (0.23 mmol) of 5-chloro-1,2,3,4-tetrahydronaphthalen-1-ol (racemate). This gave 38 mg (36% of theory, purity 77%) of the title compound.

¹H-NMR (400 MHz, CDCl₃): δ[ppm]=1.71-1.86 (m, 1H), 2.03-2.21 (m, 5H), 2.30-2.43 (m, 1H), 2.65-2.80 (m, 1H), 2.98-3.10 (m, 1H), 3.81 (s, 3H), 6.10-6.23 (m, 1H), 6.75-6.88 (m, 3H), 7.07 (s, 1H), 7.12-7.17 (m, 1H), 7.22-ca. 7.27 (m, 1H, partially under the chloroform signal)

### Example 13A

2-(4-Methoxy-2-methylphenyl)-3,5-dioxo-4-[(1R)-4-(trifluoromethyl)-2,3-dihydro-1H-inden-1-yl]-2,3,4,5-tetrahydro-1,2,4-triazine-6-carbonitrile (R enantiomer)

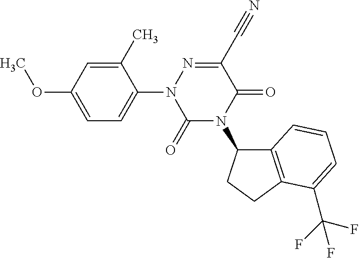 [SMILES: COc1ccc(-n2nc(C#N)c(=O)n([C@@H]3CCc4c3cccc4C(F)(F)F)c2=O)c(C)c1]

Analogously to Example 8A, 50.0 mg (0.194 mmol) of 2-(4-methoxy-2-methylphenyl)-3,5-dioxo-2,3,4,5-tetrahydro-1,2,4-triazine-6-carbonitrile from Example 2A were reacted with 52.2 mg (0.23 mmol, purity 90%) of (1S)-4-(trifluoromethyl)indan-1-ol (S enantiomer). This gave 38 mg (40% of theory, purity 90%) of the title compound.

LC-MS (Method 1): Rₜ=1.25 min., ES neg. m/z=487 (M+HCOOH—H)⁻

¹H-NMR (400 MHz, CDCl₃): δ[ppm]=2.09 (s, 3H), 2.36-2.48 (m, 1H), 2.64-2.72 (m, 1H), 3.12-3.25 (m, 1H), 3.49-3.62 (m, 1H), 3.81 (s, 3H), 6.52 (dd, 1H), 6.79-6.85 (m, 2H), 7.14 (d, 1H), 7.28-7.34 (m, 2H), 7.54 (d, 1H).

### Example 14A

3,5-Dioxo-2-[4-(2-oxoimidazolidin-1-yl)phenyl]-4-[(1R)-4-(trifluoromethyl)-2,3-dihydro-1H-inden-1-yl]-2,3,4,5-tetrahydro-1,2,4-triazine-6-carbonitrile (R enantiomer)

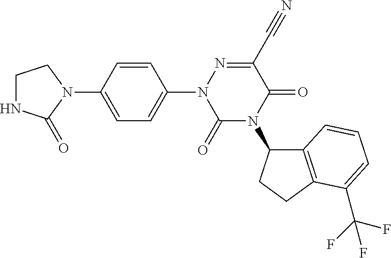 [SMILES: N#Cc1nn(-c2ccc(N3CCNC3=O)cc2)c(=O)n([C@@H]2CCc3c2cccc3C(F)(F)F)c1=O]

Analogously to Example 8A, 100.0 mg (0.34 mmol) of the compound from Example 4A were reacted with 81.4 mg (0.40 mmol) of (1S)-4-(trifluoromethyl)indan-1-ol (S enantiomer). This gave 73 mg (38% of theory, purity 85%) of the title compound.

LC-MS (Method 1): Rₜ=1.07 min., m/z=483 (M+H)⁺

¹H-NMR (400 MHz, CDCl₃): δ[ppm]=2.38-2.51 (m, 1H), 2.60-2.76 (m, 1H), 3.11-3.27 (m, 1H), 3.50-3.71 (m, 3H), 3.88-4.13 (m, 2H), 4.72 (br. s., 1H), 6.53 (dd, 1H), 7.29-7.35 (m, 2H), 7.40 (d, 2H), 7.51-7.58 (m, 1H), 7.65-7.70 (m, 2H).

### Example 15A

3,5-Dioxo-2-[4-(2-oxo-1,3-oxazolidin-3-yl)phenyl]-4-[(1R)-4-(trifluoromethyl)-2,3-dihydro-1H-inden-1-yl]-2,3,4,5-tetrahydro-1,2,4-triazine-6-carbonitrile (R enantiomer)

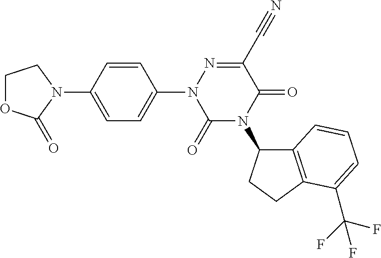 [SMILES: N#Cc1nn(-c2ccc(N3CCOC3=O)cc2)c(=O)n([C@@H]2CCc3c2cccc3C(F)(F)F)c1=O]

Analogously to Example 8A, 60.0 mg (0.20 mmol) of the compound from Example 3A were reacted with 48.6 mg (0.24 mmol) of (1S)-4-(trifluoromethyl)indan-1-ol (S enantiomer). This gave 35 mg (36% of theory) of the title compound.

LC-MS (Method 1): Rₜ=1.12 min., ES neg. m/z=528 (M+HCOOH—H)³¹

¹H-NMR (400 MHz, CDCl₃): δ[ppm]=2.39-2.50 (m, 1H), 2.63-2.72 (m, 1H), 3.14-3.26 (m, 1H), 3.52-3.64 (m, 1H), 4.09 (dd, 2H), 4.53 (dd, 2H), 6.53 (dd, 1H), 7.30-7.34 (m, 2H), 7.47 (d, 2H), 7.52-7.58 (m, 1H), 7.66-7.70 (m, 2H).

### Example 16A

2-(3-Methyl-2-oxo-2,3-dihydro-1,3-benzoxazol-6-yl)-3,5-dioxo-2,3,4,5-tetrahydro-1,2,4-triazine-6-carboxylic acid

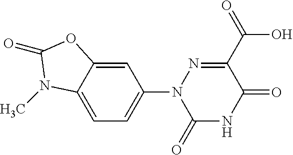 [SMILES: Cn1c(=O)oc2cc(-n3nc(C(=O)O)c(=O)[nH]c3=O)ccc21]

620 mg (2.17 mmol) of the compound from Example 5A were stirred in 6 ml of glacial acetic acid and 3 ml of conc. hydrochloric acid at reflux temperature for 2 days. After cooling to RT, the reaction mixture was diluted with 50 ml of water and, after 10 min, the solid formed was filtered off with suction. The product was dried under HV. This gave 502 mg (75% of theory) of the title compound.

¹H-NMR (400 MHz, DMSO-d₆): δ[ppm]=3.38 (s, 3H), 7.32-7.41 (m, 2H), 7.52 (d, 1H), 12.55 (br. s, 1H), 13.70 (br. s, 1H).

### Example 17A

Methyl 2-(3-methyl-2-oxo-2,3-dihydro-1,3-benzoxazol-6-yl)-3,5-dioxo-2,3,4,5-tetrahydro-1,2,4-triazine-6-carboxylate

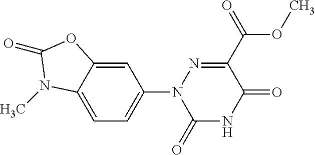 [SMILES: COC(=O)c1nn(-c2ccc3c(c2)oc(=O)n3C)c(=O)[nH]c1=O]

550 μl (7.56 mmol) of thionyl chloride were added to a suspension of 460 mg (1.51 mmol) of the compound from Example 16A in 20 ml of methanol, and the mixture was heated at reflux temperature overnight. All the volatile constituents were then removed on a rotary evaporator. The residue was triturated with a little diethyl ether, filtered off with suction and dried under HV. This gave 475 mg (99% of theory) of the title compound.

LC-MS (Method 1): Rₜ=0.58 min., m/z=319 (M+H)⁺

¹H-NMR (400 MHz, DMSO-d6): δ[ppm]=3.38 (s, 3H), 3.81 (s, 3H), 7.36 (s, 2H), 7.51 (s, 1H), 12.59 (s, 1H).

### Example 18A 5-Amino-1,3-dimethyl-1,3-dihydro-2H-benzimidazol-2-one hydrochloride

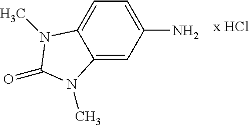

33.2 g (160 mmol) of 1,3-dimethyl-5-nitro-1,3-dihydro-2H-benzimidazol-2-one (preparation: see WO 2007/120339, Example 2, page 33) in 1790 ml of ethanol were hydrogenated in the presence of 8.8 g of palladium catalyst (10% on activated carbon, moistened with 50% water) at RT and under hydrogen standard pressure. After completion of conversion after 6 h, the catalyst was removed by filtration through kieselguhr. 45 ml of a hydrogen chloride solution (4N in dioxane) were added to the filtrate, and the mixture was concentrated to dryness on a rotary evaporator. The residue was dried further under HV. This gave 31.8 g (91% of theory) of the title compound.

LC-MS (Method 1): Rₜ=0.18 min; m/z=178 (M+H)⁺.

¹H-NMR (400 MHz, DMSO-d₆): δ[ppm]=3.33 (s, 3H), 3.34 (s, 3H), 7.06-7.15 (m, 2H), 7.23 (d, 1H), 10.29 (br.s, 3H).

### Example 19A

2-(1,3-Dimethyl-2-oxo-2,3-dihydro-1H-benzimidazol-5-yl)-3,5-dioxo-2,3,4,5-tetrahydro-1,2,4-triazine-6-carboxylic acid

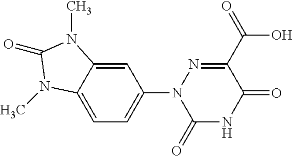 [SMILES: Cn1c(=O)n(C)c2cc(-n3nc(C(=O)O)c(=O)[nH]c3=O)ccc21]

A solution of 3.65 g (23.4 mmol) of ethyl (cyanoacetyl)carbamate in 10 ml of ethanol was added to a solution of 8.5 g (103 mmol) of sodium acetate in 25 ml of water, and the mixture was stirred at RT for 2 h. In another flask, 5.00 g (23 4 mmol) of the compound from Example 18A were suspended in 10 ml of ethanol. 15 ml of water and 3 ml of conc. hydrochloric acid were added in succession. The mixture was cooled to 0° C., and a solution of 1.62 g (23.4 mmol) of sodium nitrite in 5 ml of water was slowly added such that the temperature did not exceed 2° C. At the end of the addition, this solution was stirred at 0° C. for another 30 min and then stirred into the ethyl (cyanoacetyl)carbamate solution which had been prepared beforehand. The reaction mixture was stirred at RT overnight. The suspension formed was diluted with 80 ml of 6N aqueous hydrochloric acid and stirred for 10 min The solid was filtered off with suction, washed with a little water, stirred with 200 ml of 2-propanol and filtered off again. The solid was suspended in 100 ml of glacial acetic acid, and 2.9 g (35.1 mmol) of sodium acetate were added. The mixture was heated at reflux temperature overnight. LC-MS of a small sample showed the intermediate 2-(1,3-dimethyl-2-oxo-2,3-dihydro-1H-benzimidazol-5-yl)-3,5-dioxo-2,3,4,5-tetrahydro-1,2,4-triazine-6-carbonitrile (Method 1, Rₜ=0.62 min; m/z=299 (M+H)⁺.). The mixture was cooled slightly (to about 95° C.), 19 ml of conc. hydrochloric acid were added and the mixture was heated at reflux for 3 days, with the reaction being monitored by LC-MS. After complete hydrolysis, the mixture was allowed to cool to RT and then added to 1.5 l of ice-water. The solid formed was filtered off, washed with diethyl ether and dried under HV. This gave 4.10 g (54% of theory) of the title compound.

LC-MS (Method 6): Rₜ=0.51 min; m/z=318 (M+H)⁺.

¹H-NMR (400 MHz, DMSO-d₆): δ[ppm]=3.37 (s, 3H), 7.16-7.27 (m, 2H), 7.30 (d, 1H), 12.54 (br. s, 1H), 13.67 (br. s, 1H). (signal of one methyl group probably hidden under the water signal).

### Example 20A

2-(3-Methyl-2-oxo-2,3-dihydro-1,3-benzothiazol-6-yl)-3,5-dioxo-2,3,4,5-tetrahydro-1,2,4-triazine-6-carboxylic acid

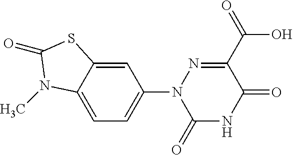 [SMILES: Cn1c(=O)sc2cc(-n3nc(C(=O)O)c(=O)[nH]c3=O)ccc21]

The title compound was prepared analogously to Example 19A from 2.50 g (13 9 mmol) of 6-amino-3-methyl-1,3-benzothiazol-2(3H)-one (J. Het. Chem. 1992, 29 (5), 1069-1076, Example 8b) and 2.17 g (13.9 mmol) of ethyl (cyanoacetyl)carbamate. Yield: 2.24 g (50% of theory).

MS (Method 7): ESpos.: m/z=321 (M+H)⁺.

### Example 21A

Methyl 2-(1,3-dimethyl-2-oxo-2,3-dihydro-1H-benzimidazol-5-yl)-3,5-dioxo-2,3,4,5-tetrahydro-1,2,4-triazine-6-carboxylate

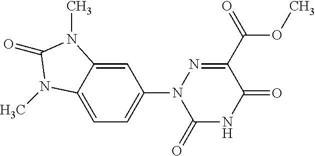 [SMILES: COC(=O)c1nn(-c2ccc3c(c2)n(C)c(=O)n3C)c(=O)[nH]c1=O]

Analogously to Example 17A, 1.86 g (5.86 mmol) of the compound from Example 19A in 75 ml of methanol were reacted with 2.13 ml (29.1 mmol) of thionyl chloride. This gave 2.0 g (94% of theory) of the title compound.

LC-MS (Method 1): Rₜ=0.54 min; m/z=331 (M+H)⁺.

¹H-NMR (400 MHz, DMSO-d₆): δ[ppm]=3.37 (s, 3H), 3.81 (s, 3H), 7.15-7.21 (m, 1H), 7.22-7.27 (m, 1H), 7.29 (d, 1H), 12.56 (s, 1H). (signal of one methyl group probably hidden under the water signal).

### Example 22A

Methyl 2-(3-methyl-2-oxo-2,3-dihydro-1,3-benzothiazol-6-yl)-3,5-dioxo-2,3,4,5-tetrahydro-1,2,4-triazine-6-carboxylate

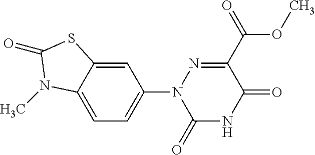 [SMILES: COC(=O)c1nn(-c2ccc3c(c2)sc(=O)n3C)c(=O)[nH]c1=O]

Analogously to Example 17A, 2.24 g (6.99 mmol) of the compound from Example 20A in 89 ml of methanol were reacted with 2.55 ml (34.9 mmol) of thionyl chloride. This gave 2.10 g (75% of theory, purity 83%) of the title compound.

LC-MS (Method 1): Rₜ=0.69 min; m/z=335 (M+H)⁺.

¹H-NMR (400 MHz, DMSO-d₆): δ[ppm]=3.44 (s, 3H), 3.81 (s, 3H), 7.43 (d, 1H), 7.52 (dd, 1H), 7.82 (d, 1H), 12.60 (br. s, 1H).

### WORKING EXAMPLES

### Example 1

3,5-Dioxo-2-[4-(2-oxo-1,3-oxazolidin-3-yl)phenyl]-4-[(1R)-4-(trifluoromethyl)-2,3-dihydro-1H-inden-1-yl]-2,3,4,5-tetrahydro-1,2,4-triazine-6-carboxylic acid (R enantiomer)

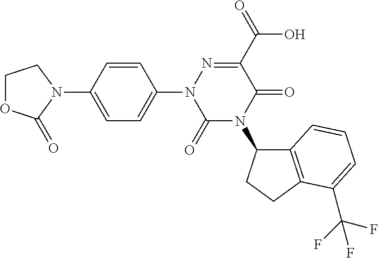 [SMILES: O=C(O)c1nn(-c2ccc(N3CCOC3=O)cc2)c(=O)n([C@@H]2CCc3c2cccc3C(F)(F)F)c1=O]

32 mg (66 μmol) of the compound from Example 15A in 2 ml of glacial acetic acid and 1 ml of conc. hydrochloric acid were heated at reflux temperature for 1 h. After cooling to RT, the entire reaction mixture was separated by preparative HPLC (Method 5). This gave 22 mg (66% of theory) of the title compound.

LC-MS (Method 1): Rₜ=0.94 min; m/z=503 (M+H)⁺.

¹H-NMR (400 MHz, CDCl₃): δ[ppm]=2.43-2.55 (m, 1H), 2.64-2.76 (m, 1H), 3.16-3.30 (m, 1H), 3.53-3.66 (m, 1H), 4.05-4.13 (m, 2H), 4.49-4.57 (m, 2H), 6.60 (dd, 1H), 7.30-7.38 (m, 2H), 7.49-7.61 (m, 3H), 7.68 (d, 2H).

The following compounds in Table 1 (Examples 2 to 8) were prepared analogously to Example 1 from the corresponding precursors, where the reaction time was determined by monitoring the reaction by HPLC or LC-MS. All LC-MS data given in Table 1 were measured according to Method 1.

TABLE 1

| | IUPAC name/structure | | |
|---|---|---|---|
| Example | (Yield) | Precursor | Analytical data |
| 2 | 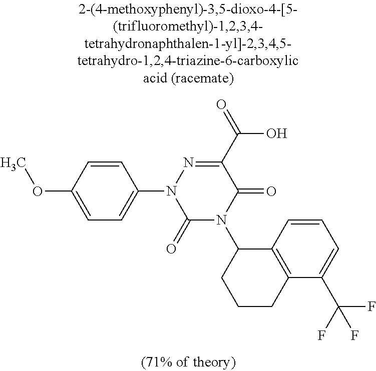 [SMILES: COc1ccc(-n2nc(C(=O)O)c(=O)n(C3CCCc4c3cccc4C(F)(F)F)c2=O)cc1] | Ex. 8A | LC-MS: Rₜ = 1.10 min., m/z = 462 (M + H)⁺. ¹H-NMR (400 MHz, CDCl₃): δ [ppm] = 1.76- 1.90 (m, 1H), 2.14- 2.27 (m, 2H), 2.34- 2.49 (m, 1H), 2.90- 3.03 (m, 1H), 3.08- 3.19 (m, 1H), 3.84 (s, 3H), 6.25-6.35 (m, 1H), 6.96 (d, 2H), 7.12 (d, 1H), 7.21-7.27 (m, 1H, partially under the chloroform signal) 7.41 (br. d, 2H), 7.53-7.58 (m, 1H). |
| 3 | 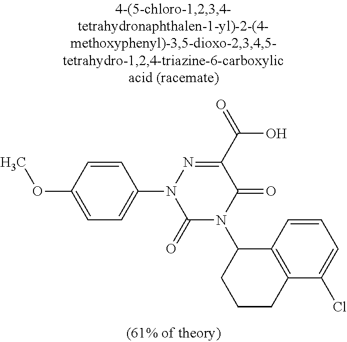 [SMILES: COc1ccc(-n2nc(C(=O)O)c(=O)n(C3CCCc4c(Cl)cccc43)c2=O)cc1] | Ex. 9A | LC-MS: Rₜ = 1.08 min, ES neg. m/z = 426 (M − H)⁻ ¹H-NMR (400 MHz, CDCl₃): δ [ppm] = 1.75- 1.90 (m, 1H), 2.11- 2.28 (m, 2H), 2.36- 2.50 (m, 1H), 2.70- 2.83 (m, 1H), 3.08 (br. d, 1H), 3.84 (s, 3H), 6.20-6.30 (m, 1H), 6.84 (d, 1H), 6.96 (br. d, 2H), 7.09 (t, 1H), 7.28 (d, 1H, partially under the chloroform signal) 7.41 (br. d, 2H). |
| 4 | 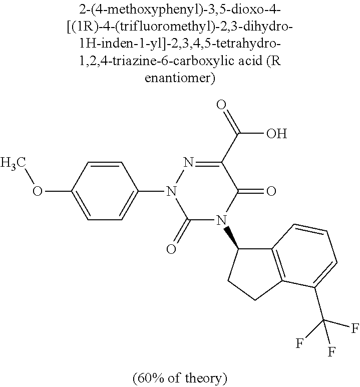 [SMILES: COc1ccc(-n2nc(C(=O)O)c(=O)n([C@@H]3CCc4c3cccc4C(F)(F)F)c2=O)cc1] | Ex. 10A | LC-MS: Rₜ = 1.06 min., m/z = 448 (M + H)⁺. ¹H-NMR (400 MHz, CDCl₃): δ [ppm] = 2.43- 2.55 (m, 1H), 2.64- 2.76 (m, 1H), 3.17- 3.29 (m, 1H), 3.58 (dd, 1H), 3.84 (s, 3H), 6.61 (dd, 1H), 6.97 (d, 2H), 7.31-7.35 (m, 2H), 7.41 (d, 2H), 7.54-7.61 (m, 1H). |
| 5 | 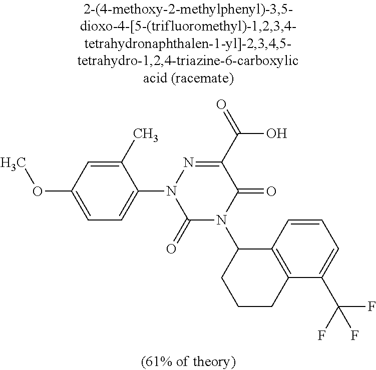 [SMILES: COc1ccc(-n2nc(C(=O)O)c(=O)n(C3CCCc4c3cccc4C(F)(F)F)c2=O)c(C)c1] | Ex. 11A | LC-MS: Rₜ = 1.14 min., m/z = 476 (M + H)⁺. ¹H-NMR (400 MHz, CDCl₃): δ [ppm] = 1.76- 1.89 (m, 1H), 2.08 (br. s., 3H), 2.15-2.28 (m, 2H), 2.34-2.47 (m, 1H), 2.88-3.01 (m, 1H), 3.06-3.18 (m, 1H), 3.80 (s, 3H), 6.25- 6.34 (m, 1H), 6.76- 6.84 (m, 2H), 7.10 (d, 1H), 7.18 (d, 1H), 7.21- 7.25 (m, 1H), 7.54 (d, 1H). |
| 6 | 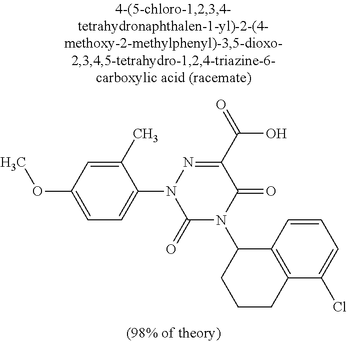 [SMILES: COc1ccc(-n2nc(C(=O)O)c(=O)n(C3CCCc4c(Cl)cccc43)c2=O)c(C)c1] | Ex. 12A | LC-MS: Rₜ = 1.11 min., m/z = 442 (M + H)⁺. ¹H-NMR (400 MHz, CDCl₃): δ [ppm] = 1.73- 1.89 (m, 1H), 2.09 (br. s., 3H), 2.14-2.25 (m, 2H), 2.35-2.48 (m, 1H), 2.68-2.81 (m, 1H), 3.06 (br. d, 1H), 3.81 (s, 3H), 6.24 (dd, 1H), 6.74-6.86 (m, 3H), 7.08 (t, 1H), 7.18 (d, 1H), 7.27-7.29 (m, 1H, partially under the CHCl₃ signal) |
| 7 | 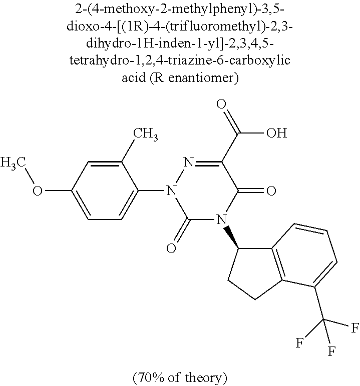 [SMILES: COc1ccc(-n2nc(C(=O)O)c(=O)n([C@@H]3CCc4c3cccc4C(F)(F)F)c2=O)c(C)c1] | 13A | LC-MS: Rₜ = 1.06 min., m/z = 462 (M + H)⁺. ¹H-NMR (400 MHz, CDCl₃): δ [ppm] = 2.10 (br. s, 3H), 2.42-2.54 (m, 1H), 2.65-2.77 (m, 1H), 3.16-3.29 (m, 1H), 3.52-3.64 (m, 1H), 3.81 (s, 3H), 6.60 (dd, 1H), 6.77-6.84 (m, 2H), 7.18 (d, 1H), 7.28- 7.37 (m, 2H), 7.56 (d, 1H). |
| 8 | 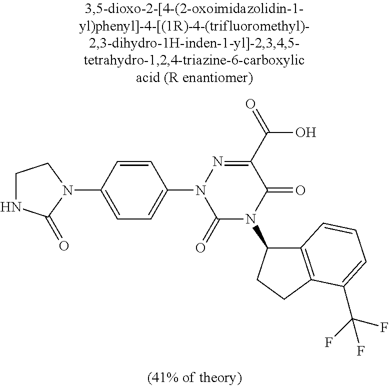 [SMILES: O=C(O)c1nn(-c2ccc(N3CCNC3=O)cc2)c(=O)n([C@@H]2CCc3c2cccc3C(F)(F)F)c1=O] | 14A | LC-MS: Rₜ = 0.91 min., m/z = 502 (M + H)⁺. ¹H-NMR (400 MHz, CDCl₃): δ [ppm] = 2.44- 2.55 (m, 1H), 2.64- 2.76 (m, 1H), 3.16- 3.30 (m, 1H), 3.53- 3.67 (m, 3H), 3.96 (dd, 2H), 4.90 (br. s, 1H), 6.60 (dd, 1H), 7.30- 7.37 (m, 2H), 7.47 (d, 2H), 7.53-7.60 (m, 1H), 7.67 (d, 2H). |

### Example 9

Methyl 2-(1,3-dimethyl-2-oxo-2,3-dihydro-1H-benzimidazol-5-yl)-3,5-dioxo-4-1[(1R)-4-(trifluoromethyl)-2,3-dihydro-1H-inden-1-yl]-2,3,4,5-tetrahydro-1,2,4-triazine-6-carboxylate (R enantiomer)

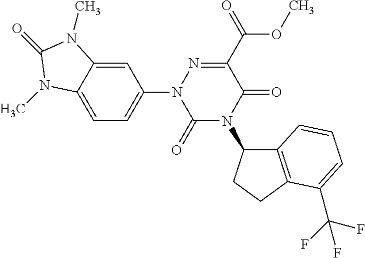 [SMILES: COC(=O)c1nn(-c2ccc3c(c2)n(C)c(=O)n3C)c(=O)n([C@@H]2CCc3c2cccc3C(F)(F)F)c1=O]

100 mg (302 μmol) of the compound from Example 21A and 79.3 mg (392 μmol) of (1S)-4-(trifluoromethyl)indan-1-ol and 261.3 mg (1 mmol) of triphenylphosphine were initially charged in 3 ml of THF and 3 ml of DMF. 89 μl (453 μmol) of DIAD were added dropwise and the mixture was stirred at RT for 2 h. The entire reaction mixture was then separated by preparative HPLC (Method 5). This gave 85 mg (55% of theory) of the title compound.

LC-MS (Method 1): Rₜ=1.07 min., m/z=516 (M+H)⁺.

¹H-NMR (400 MHz, CD₂Cl₂): δ[ppm]=2.36-2.51 (m, 1H), 2.57-2.72 (m, 1H), 3.11-3.24 (m, 1H), 3.39 (s, 3H), 3.41 (s, 3H), 3.46-3.58 (m, 1H), 3.91 (s, 3H), 6.55 (dd, 1H), 6.99-7.08 (m, 2H), 7.13-7.18 (m, 1H), 7.29-7.40 (m, 2H), 7.54 (d, 1H).

### Example 10

Methyl 2-(3-methyl-2-oxo-2,3-dihydro-1,3-benzothiazol-6-yl)-3,5-dioxo-4-[(1R)-4-(trifluoromethyl)-2,3-dihydro-1H-inden-1-yl]-2,3 ,4,5-tetrahydro-1,2,4-triazine-6-carboxylate (R enantiomer)

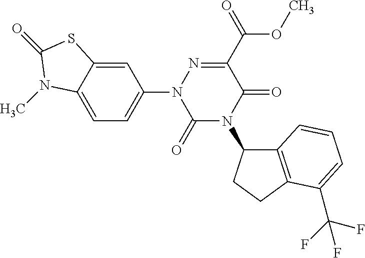 [SMILES: COC(=O)c1nn(-c2ccc3c(c2)sc(=O)n3C)c(=O)n([C@@H]2CCc3c2cccc3C(F)(F)F)c1=O]

Analogously to Example 9, 100 mg (0.29 mmol) of the compound from Example 22A were reacted with 156 mg (598 μmol) of triphenylphosphine, 106 μl (538 μmol) of DIAD and 66.5 mg (0.33 mmol) of (1S)-4-(trifluoromethyl)indan-1-ol (S enantiomer). This gave 50 mg (30% of theory) of the title compound.

LC-MS (Method 1): Rₜ=1.17 min., m/z=519 (M+H)⁺.

¹H-NMR (400 MHz, CD₂Cl₂): δ[ppm]=2.38-2.50 (m, 1H), 2.58-2.71 (m, 1H), 3.12-3.25 (m, 1H), 3.45 (s, 3H), 3.43-3.58 (m, 1H), 3.91 (s, 3H), 6.54 (dd, 1H), 7.12 (d, 1H), 7.28-7.39 (m, 2H), 7.46 (dd, 1H), 7.54 (d, 1H), 7.58 (d, 1H).

### Example 11

Methyl 3,5-dioxo-2-[4-(2-oxo-1,3-oxazolidin-3-yl)phenyl]-4-[5-(trifluoromethyl)-1,2,3,4-tetrahydronaphthalen-1-yl]-2,3,4,5-tetrahydro-1,2,4-triazine-6-carboxylate (racemate)

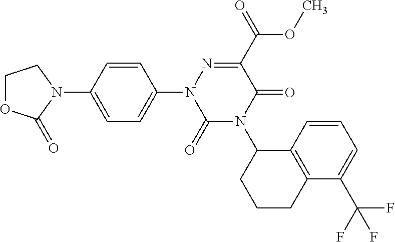 [SMILES: COC(=O)c1nn(-c2ccc(N3CCOC3=O)cc2)c(=O)n(C2CCCc3c2cccc3C(F)(F)F)c1=O]

150 mg (451 μmol) of the compound from Example 7A and 117.1 mg (542 μmol) of 5-(trifluoromethyl)-1,2,3,4-tetrahydronaphthalen-1-ol (racemate) and 201.3 mg (767 μmol) of triphenylphosphine were dissolved in 3.1 ml of THF and 6.2 ml of DMF. 142 μl (722 μmol) of DIAD were added dropwise and the mixture was stirred at RT for 2 h. The entire reaction mixture was then separated by preparative HPLC (Method 5). This gave 102 mg (43% of theory) of the title compound.

LC-MS (Method 1): Rₜ=1.15 min., m/z=531 (M+H)⁺.

¹H-NMR (400 MHz, CD₂Cl₂): δ[ppm]=1.73-1.88 (m, 1H), 2.11-2.23 (m, 2H), 2.31-2.44 (m, 1H), 2.88-3.00 (m, 1H), 3.05-3.15 (m, 1H), 3.91 (s, 3H), 4.03-4.09 (m, 2H), 4.45-4.52 (m, 2H), 6.18-6.27 (m, 1H), 7.18-7.27 (m, 2H), 7.46-7.55 (m, 3H), 7.66 (d, 2H).

### Example 12

Methyl 4-(5-chloro-1,2,3,4-tetrahydronaphthalen-1-yl)-3,5-dioxo-2-[4-(2-oxo-1,3-oxazolidin-3-yl)phenyl]-2,3,4,5-tetrahydro-1,2,4-triazine-6-carboxylate (racemate)

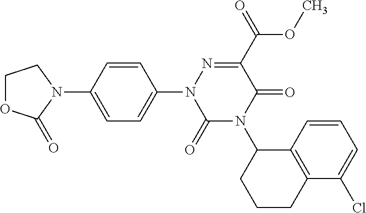 [SMILES: COC(=O)c1nn(-c2ccc(N3CCOC3=O)cc2)c(=O)n(C2CCCc3c(Cl)cccc32)c1=O]

Analogously to Example 11, 150 mg (0.45 mmol) of the compound from Example 7A were reacted under Mitsunobu conditions with 90.9 mg (0.54 mmol) of 5-chloro-1,2,3,4-tetrahydronaphthalen-1-ol. This gave 140 mg (62% of theory) of the title compound.

LC-MS (Method 1): Rₜ=1.13 min., m/z=497 (M+H)⁺.

¹H-NMR (400 MHz, CD₂Cl₂): δ[ppm]=1.72-1.88 (m, 1H), 2.06-2.21 (m, 3H), 2.33-2.46 (m, 1H), 2.68-2.80 (m, 1H), 2.99-3.09 (m, 1H), 3.91 (s, 3H), 4.03-4.09 (m, 2H), 4.49 (t, 2H), 6.12-6.23 (m, 1H), 6.92 (d, 1H), 7.08 (t, 1H), 7.25 (d, 1H), 7.50 (d, 2H), 7.66 (d, 2H).

### Example 13

Methyl 4-(5-chloro-1,2,3,4-tetrahydronaphthalen-1-yl)-2-(3-methyl-2-oxo-2,3-dihydro-1,3-benzoxazol-6-yl)-3,5-dioxo-2,3,4,5-tetrahydro-1,2,4-triazine-6-carboxylate (racemate)

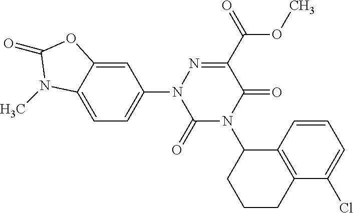 [SMILES: COC(=O)c1nn(-c2ccc3c(c2)oc(=O)n3C)c(=O)n(C2CCCc3c(Cl)cccc32)c1=O]

Analogously to Example 11, 150 mg (0.47 mmol) of the compound from Example 17A, 210 mg (801 μmol) of triphenylphosphine and 148 μl (754 μmol) of DIAD were reacted with 103.3 mg (570 μmol) of 5-chloro-1,2,3,4-tetrahydronaphthalen-1-ol (racemate). This gave 140 mg (62% of theory) of the title compound.

LC-MS (Method 1): Rₜ=1.14 min., m/z=483 (M+H)⁺.

¹H-NMR (400 MHz, CD₂Cl₂): δ[ppm]=1.73-1.88 (m, 1H), 2.06-2.22 (m, 1H), 2.31-2.45 (m, 1H), 2.67-2.79 (m, 1H), 3.04 (br. d, 1H), 3.41 (s, 3H), 3.92 (s, 3H), 6.11-6.23 (m, 1H), 6.92 (d, 1H), 7.01-7.12 (m, 2H), 7.26 (d, 1H), 7.31-7.44 (m, 2H).

### Example 14

Methyl 2-(3-methyl-2-oxo-2,3-dihydro-1,3-benzoxazol-6-yl)-3,5-dioxo-4-[5-(trifluoromethyl)-1,2,3,4-tetrahydronaphthalen-1-yl]-2,3,4,5-tetrahydro-1,2,4-triazine-6-carboxylate (racemate)

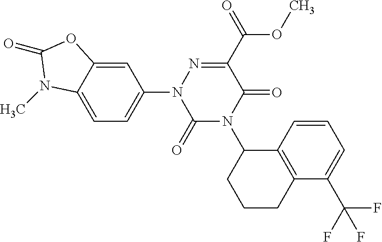 [SMILES: COC(=O)c1nn(-c2ccc3c(c2)oc(=O)n3C)c(=O)n(C2CCCc3c2cccc3C(F)(F)F)c1=O]

Analogously to Example 11, 150 mg (0.47 mmol) of the compound from Example 17A, 210 mg (801 μmol) of triphenylphosphine and 148 μl (754 μmol) of DIAD were reacted with 122.3 mg (570 μmol) of 5-(trifluoromethyl)-1,2,3,4-tetrahydronaphthalen-1-ol (racemate). This gave 135 mg (55% of theory) of the title compound.

LC-MS (Method 1): Rₜ=1.16 min., m/z=517 (M+H)⁺.

¹H-NMR (400 MHz, CD₂Cl₂): δ[ppm]=1.73-1.88 (m, 1H), 2.12-2.23 (m, 2H), 2.30-2.44 (m, 1H), 2.87-2.99 (m, 1H), 3.04-3.15 (m, 1H), 3.41 (s, 3H), 3.92 (s, 3H), 6.18-6.27 (m, 1H), 7.04 (d, 1H), 7.18-7.27 (m, 2H), 7.31-7.38 (m, 2H), 7.53 (d, 1H).

### Example 15

3,5-Dioxo-2-[4-(2-oxo-1,3-oxazolidin-3-yl)phenyl]-4-[5-(trifluoromethyl)-1,2,3,4-tetrahydronaphthalen-1-yl]-2,3,4,5-tetrahydro-1,2,4-triazine-6-carboxylic acid (racemate)

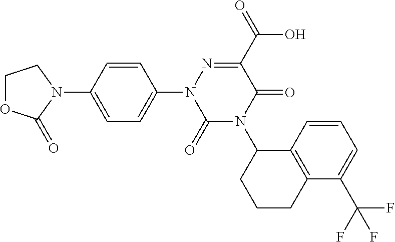 [SMILES: O=C(O)c1nn(-c2ccc(N3CCOC3=O)cc2)c(=O)n(C2CCCc3c2cccc3C(F)(F)F)c1=O]

90 mg (0.17 mmol) of the compound from Example 11 in 3 ml of glacial acetic acid/conc. hydrochloric acid 2:1 (v/v) were heated at reflux temperature for 2 h. After cooling to RT, the mixture was diluted with 2.5 ml of DMSO and 2.5 ml of acetonitrile and separated directly by preparative HPLC (Method 5). This gave 59 mg (67% of theory) of the title compound.

LC-MS (Method 1): Rₜ=1.16 min., m/z=517 (M+H)⁺.

¹H-NMR (400 MHz, CD₂Cl₂): δ[ppm]=1.76-1.89 (m, 1H), 2.14-2.27 (m, 2H), 2.33-2.44 (m, 1H), 2.90-3.01 (m, 1H), 3.13 (d, 1H), 4.03-4.11 (m, 2H), 4.49 (dd, 2H), 6.25-6.34 (m, 1H), 7.15-7.21 (m, 1H), 7.23-7.30 (m, 1H), 7.51-7.58 (m, 3H), 7.69 (d, 2H), 11.93 (br.s, 1H).

The following compounds of Table 2 (Examples 16 to 19) were prepared analogously to Example 1 from the corresponding precursors under acidic hydrolysis conditions:

TABLE 2
(All LC-MS data were measured according to Method 1).

| | IUPAC name/structure | | |
|---|---|---|---|
| Example | (Yield) | Precursor | Analytical data |
| 16 | 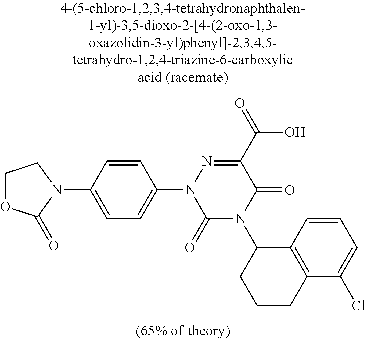 [SMILES: O=C(O)c1nn(-c2ccc(N3CCOC3=O)cc2)c(=O)n(C2CCCc3c(Cl)cccc32)c1=O] | Ex. 12 | LC-MS: Rₜ = 0.99 min., m/z = 483 (M + H)⁺. ¹H-NMR (500 MHz, CD₂Cl₂): δ [ppm] = 1.76- 1.89 (m, 1H), 2.12-2.25 (m, 1H), 2.34-2.46 (m, 1H), 2.70-2.81 (m, 1H), 3.07 (br. d, 1H), 4.03-4.11 (m, 2H), 4.49 (dd, 2H), 6.25 (dd, 1H), 6.90 (d, 1H), 7.10 (t, 1H), 7.29 (d, 1H), 7.55 (br. d, 2H), 7.69 (d, 2H), 12.0 (br.s, 1H). |
| 17 | 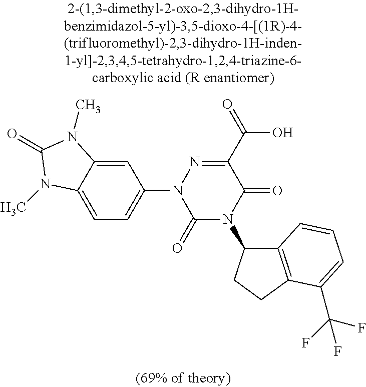 [SMILES: Cn1c(=O)n(C)c2cc(-n3nc(C(=O)O)c(=O)n([C@@H]4CCc5c4cccc5C(F)(F)F)c3=O)ccc21] | Ex. 9 | LC-MS: Rₜ = 0.92 min., m/z = 502 (M + H)⁺. ¹H-NMR (500 MHz, CD₂Cl₂): δ [ppm] = 2.43- 2.54 (m, 1H), 2.65-2.77 (m, 1H), 3.16-3.28 (m, 1H), 3.40 (s, 3H), 3.42 (s, 3H), 3.49-3.61 (m, 1H), 6.61 (dd, 1H), 7.05 (d, 1H), 7.11 (s, 1H), 7.17-7.24 (m, 1H), 7.32-7.42 (m, 2H), 7.58 (d, 1H). |
| 18 | 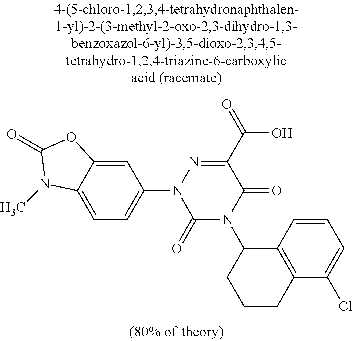 [SMILES: Cn1c(=O)oc2cc(-n3nc(C(=O)O)c(=O)n(C4CCCc5c(Cl)cccc54)c3=O)ccc21] | Ex. 13 | LC-MS: Rₜ = 2.29 min., m/z = 469 (M + H)⁺. ¹H-NMR (500 MHz, CD₂Cl₂): δ [ppm] = 1.76- 1.89 (m, 1H), 2.13-2.24 (m, 2H), 2.34-2.45 (m, 1H), 2.70-2.81 (m, 1H), 3.07 (br. d, 1H), 3.42 (s, 3H), 6.25 (dd, 1H), 6.90 (d, 1H), 7.04-7.13 (m, 2H), 7.29 (d, 1H), 7.40 (d, 2H), 12.00 (br.s, 1H). |
| 19 | 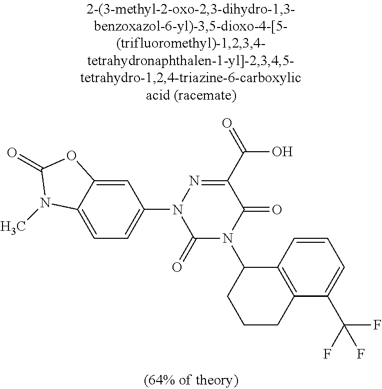 [SMILES: Cn1c(=O)oc2cc(-n3nc(C(=O)O)c(=O)n(C4CCCc5c4cccc5C(F)(F)F)c3=O)ccc21] | Ex. 14 | LC-MS: Rₜ = 1.02 min., m/z = 503 (M + H)⁺. ¹H-NMR (500 MHz, CD₂Cl₂): δ [ppm] = 1.76- 1.89 (m, 1H), 2.14-2.27 (m, 1H), 2.32-2.44 (m, 1H), 2.89-3.00 (m, 1H) , 3.12 (d, 1H), 3.41 (s, 1H), 6.25-6.33 (m, 1H), 7.07 (d, 1H), 7.16-7.21 (m, 1H), 7.23-7.29 (m, 1H), 7.34-7.44 (m, 1H), 7.56 (d, 1H). |

### Example 20

2-(3-Methyl-2-oxo-2,3-dihydro-1,3-benzothiazol-6-yl)-3,5-dioxo-4-[(1R)-4-(trifluoromethyl)-2,3-dihydro-1H-inden-1-yl]-2,3,4,5-tetrahydro-1,2,4-triazine-6-carboxylic acid (R enantiomer)

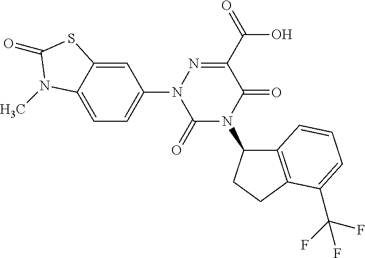 [SMILES: Cn1c(=O)sc2cc(-n3nc(C(=O)O)c(=O)n([C@@H]4CCc5c4cccc5C(F)(F)F)c3=O)ccc21]

95 mg (0.18 mmol) of the compound from Example 10 in 1.9 ml of glacial acetic acid/conc. hydrochloric acid 2:1 (v/v) were heated at reflux temperature for 2 h. After cooling to RT, the mixture was diluted with 50 ml of water and stirred vigorously for 5 min The solid formed was filtered off with suction, washed with diethyl ether and dried under HV. This gave 58 mg (63% of theory) of the title compound.

LC-MS (Method 1): Rₜ=0.99 min., m/z=505 (M+H)⁺.

¹H-NMR (400 MHz, CD₂Cl₂): δ[ppm]=2.33-2.44 (m, 1H), 2.57-2.69 (m, 1H), 3.09-3.20 (m, 1H), 3.38 (s, 3H), 3.46 (dd, 1H), 6.52 (dd, 1H), 7.07 (d, 1H), 7.24-7.33 (m, 2H), 7.43 (dd, 1H), 7.47-7.53 (m, 1H), 7.56 (d, 1H).

B. Assessment of Pharmacological Efficacy

The pharmacological activity of the compounds according to the invention can be shown in the assays described below:

Abbreviations:

| | |
|---|---|
| Abz-HPFHL- | 1-[N-(3-aminobenzoyl)histidylprolyl- |
| Lys(Dnp)—NH₂ | phenylalanylhistidylleucyl-N⁶- |
| | (2,4-dinitrophenyl)lysine |
| AMC | 7-amido-4-methylcoumarin |
| BNP | brain natriuretic peptide |
| BSA | bovine serum albumin |
| CHAPS | 3-[(3-cholamidopropyl)dimethylammonio]- |
| | 1-propanesulfonate |
| HEPES | N-(2-hydroxyethyl)piperazine-N′-2-ethanesulfonic |
| | acid |
| IC | inhibition concentration |
| MeOSuc | methoxysuccinyl |
| NADP | nicotinamide adenine dinucleotide phosphate |
| PBS | phosphate-buffered saline solution |
| PEG | polyethylene glycol |
| v/v | volume to volume ratio (of a solution) |
| w/v | weight to volume ratio (of a solution) |

B-1. Enzymatic Chymase Assay

The enzyme source used is recombinant human chymase (expressed in HEK293 cells) or chymase purified from hamsters' tongues. The substrate used for chymase is Abz-HPFHL-Lys(Dnp)-NH₂. For the assay, 1 μl of a 50-fold concentrated solution of test substance in DMSO, 24 μl of enzyme solution (dilution 1:80 000 human or 1:4000 hamster) and 25 μl of substrate solution (final concentration 10 μM) in assay buffer (Tris 50 mM (pH 7.5), sodium chloride 150 mM, BSA 0.10%, Chaps 0.10%, glutathione 1 mM, EDTA 1 mM) are combined in a white 384-hole microtitre plate (Greiner Bio-One, Frickenhausen, Germany) The reaction is incubated at 32 degrees for 60 min and the fluorescence emission at 465 nm after excitation at 340 nm is measured in a fluorescence reader, for example Tecan Ultra (Tecan, Männedorf, Switzerland).

One test compound is tested on the same microtitre plate in 10 different concentrations from 30 μM to 1 nM in a double determination. The data are normalized (enzyme reaction without inhibitor=0% inhibition, all assay components without enzyme=100% inhibition) and IC₅₀ values are calculated using in-house software. Compounds in the context of the invention which were tested in this assay inhibited chymase activity with an IC₅₀ of less than 10 μM.

IC₅₀ values representative of the compounds of the invention are shown in Table 3 below:

| | hamster chymase |
|---|---|
| Example No.: | IC₅₀ [μM] |
| 1 | 0.12 |
| 2 | 0.4 |
| 3 | 2.3 |
| 4 | 0.82 |
| 5 | 0.5 |
| 6 | 2.3 |
| 7 | 0.43 |
| 8 | 0.24 |
| 9 | 0.0065 |
| 10 | 0.018 |
| 11 | 0.21 |
| 12 | 0.52 |
| 13 | 0.12 |
| 14 | 0.031 |
| 15 | 0.041 |
| 16 | 0.19 |
| 17 | 0.0054 |
| 18 | 0.064 |
| 19 | 0.0043 |
| 20 | 0.0056 |

B-2. Measurement of Contraction on Isolated Aorta Rings from Hamsters

Male Syrian hamsters (120-150 g) were euthanized with carbon dioxide. The aorta was prepared and placed into ice-cold Krebs-Henseleit buffer. (Composition in mmol/1: sodium chloride 112, potassium chloride 5.9, calcium chloride 2.0, magnesium chloride 1.2, sodium dihydrogenphosphate 1.2, sodium hydrogencarbonate 25, glucose 11.5). The aorta was cut into rings of length 2 mm, transferred to an organ bath filled with 5 ml of Krebs-Henseleit buffer and connected to a myograph (DMT, Denmark). The buffer was warmed to 37° C. and sparged with 95% oxygen, 5% carbon dioxide. In order to measure the isometric muscle contraction, the aorta rings were mounted between two hooks. One of the hooks was connected to a pressure transducer. The second hook was movable and allowed precise setting of the initial load by a protocol described by Mulvany and Halpern (Circulation Research 1977; 41:19-26).

Before each experiment, the responsiveness of the preparation was tested by adding potassium-containing Krebs-Henseleit solution (50 mmol/l KCl). A synthetic peptide, angiotensin 1-18, was used to induce contraction of the aorta rings. The angiotensin 1-18 is converted to angiotensin II independently of ACE. Subsequently, the aorta rings were incubated with the test substance for 20 min and the contraction measurement was repeated. Chymase inhibition is shown as a reduction in the contraction induced by angiotensin 1-18.

B-3. Isoprenaline-induced Cardiac Fibrosis Model in Hamsters

For the experiments, male Syrian hamsters having a body weight of 130-160 g were used. Cardiac hypertrophy and cardiac fibrosis were induced by a daily subcutaneous injection of 20 mg/kg isoprenaline over 7 days. The test substance was administered orally to the animals 2 hours before the injection of the isoprenaline. Control groups were treated subcutaneously and orally with solvents in a corresponding manner. At the end of the experiment, the hearts were removed, weighed and fixed. The fibrotic tissue on the histological sections from the hearts was marked with the aid of Sirius Red staining Subsequently, the fibrotic area was determined by planimetry.

C. Working Examples for Pharmaceutical Compositions

The compounds of the invention can be converted to pharmaceutical formulations as follows:

Tablet:

Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

Production:

The mixture of compound of the invention, lactose and starch is granulated with a 5% solution (w/w) of the PVP in water. The granules are dried and then mixed with the magnesium stearate for 5 minutes. This mixture is compressed in a conventional tabletting press (see above for format of the tablet). The guide value used for the pressing is a pressing force of 15 kN.

Suspension for Oral Administration:

Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

Production:

The Rhodigel is suspended in ethanol; the compound of the invention is added to the suspension.

The water is added while stirring. The mixture is stirred for about 6 h before swelling of the Rhodigel is complete.

Solution for Oral Administration:

Composition:

500 mg of the compound of the invention, 2.5 g of polysorbate and 97 g of polyethylene glycol 400. 20 g of oral solution correspond to a single dose of 100 mg of the compound of the invention.

Production:

The compound of the invention is suspended in the mixture of polyethylene glycol and polysorbate with stirring. The stirring operation is continued until dissolution of the compound of the invention is complete.

I.V. Solution:

The compound of the invention is dissolved in a concentration below the saturation solubility in a physiologically acceptable solvent (e.g. isotonic saline solution, glucose solution 5% and/or PEG 400 solution 30%). The solution is subjected to sterile filtration and dispensed into sterile and pyrogen-free injection vessels.

1. A compound of the formula (I) 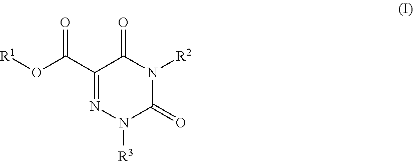
in which
R¹ represents hydrogen or (C₁-C₄)-alkyl,
R² represents a group of the formula 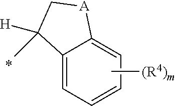
where
* represents the point of attachment to the triazinedione nitrogen atom,
A represents —CH₂—, —CH₂—CH₂—, —O—CH₂—** or oxygen,
in which ** represents the point of attachment to the phenyl ring,
m represents a number 0, 1 or 2,
R⁴ represents hydrogen, halogen, difluoromethyl, trifluoromethyl, (C₁-C₄)-alkyl, difluoromethoxy, trifluoromethoxy or (C₁-C₄)-alkoxy,
R³ represents 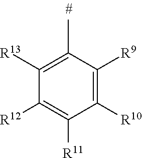
where
# represents the point of attachment to the triazinedione nitrogen atom,
R⁹ represents hydrogen,
R¹⁰ represents hydrogen, halogen, (C₁-C₄)-alkyl or (C₁-C₄)-alkoxy,
R¹¹ represents (C₁-C₄)alkyl, (C₁-C₄)-alkoxy or —N(R¹⁴R¹⁵), in which (C₁-C₄)-alkyl may be up to trisubstituted by halogen,
in which (C₁-C₄)-alkoxy may be substituted by a substituent selected from the group consisting of hydroxy, (C₁-C₄)-alkoxycarbonyl, amino, mono-(C₁-C₄)-alkylamino, di-(C₁-C₄)-alkylamino, aminocarbonyl, mono-(C₁-C₄)-alkyl aminocarbonyl and di-(C₁-C₄)-alkyl aminocarbonyl,
where
R¹⁴ represents (C₁-C₄)-alkyl, (C₁-C₄)-alkoxycarbonyl or (C₁-C₄)-alkylaminocarbonyl,
in which (C₁-C₄)-alkylaminocarbonyl may be substituted by hydroxy or (C₁-C₄)-alkoxy,
R¹⁵ represents hydrogen or (C₁-C₄)-alkyl,
or
R¹¹ represents 4- to 7-membered heterocyclyl or 5- to 6-membered heteroaryl,
in which 4- to 7-membered heterocyclyl may be substituted by 1 to 3 sub stituents independently of one another selected from the group consisting of halogen, trifluoromethyl, (C₁-C₄)-alkyl, hydroxy, oxo, amino and (C₁-C₄)-alkoxycarbonyl,
in which 5- to 6-membered heteroaryl may be substituted by 1 or 2 sub stituents independently of one another selected from the group consisting of halogen, trifluoromethyl, (C₁-C₄)-alkyl, hydroxy, amino and (C₁-C₄)-alkoxycarbonyl,
R¹² represents hydrogen, halogen, cyano, (C₁-C₄)-alkyl or (C₁-C₄)-alkoxy,
R¹³ represents hydrogen, halogen, (C₁-C₄)-alkyl or (C₁-C₄)-alkoxy,
or
R³ represents 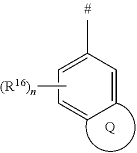
where
# represents the point of attachment to the triazinedione nitrogen atom, the ring Q represents 5- to 7-membered heterocyclyl or 5- or 6-membered heteroaryl,
in which 5- to 7-membered heterocyclyl and 5- or 6-membered heteroaryl may be substituted by 1 to 4 substituents independently selected from the group of halogen, difluoromethyl, trifluoromethyl, trideuteromethyl, (C₁-C₆)-alkyl, (C₃-C₇)-cycloalkyl, oxo, hydroxyl, (C₁-C₄)-alkylcarbonyl, (C₁-C₄)-alkoxycarbonyl, aminocarbonyl and (C₁-C₄)-alkyl sulfonyl,
in which (C₁-C₆)-alkyl and (C₃-C₇)-cycloalkyl may in turn be substituted by 1 to 3 substituents independently selected from the group of halogen, cyano, trifluoromethyl, (C₃-C₇)-cycloalkyl, hydroxyl, (C₁-C₄)-alkoxy and 4- to 7-membered heterocyclyl,
and
in which two (C₁-C₆)-alkyl radicals attached to a carbon atom of 5- to 7-membered heterocyclyl together with the carbon atom to which they are attached may form a 3- to 6-membered carbocycle,
R¹⁶ represents halogen, (C₁-C₄)-alkyl or (C₁-C₄)-alkoxy,
n represents a number 0, 1, 2 or 3,
and the salts, solvates and solvates of the salts thereof.

2. The compound of claim 1 of the formula (I), in which
R¹ represents hydrogen or (C₁-C₄)-alkyl,
R² represents a group of the formula 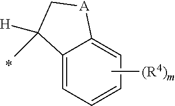
where
* represents the point of attachment to the triazinedione nitrogen atom,
A represents —CH₂— or —CH₂—CH₂—,
m represents a number 0, 1 or 2,
R⁴ represents hydrogen, fluorine, chlorine, difluoromethyl, trifluoromethyl or methyl,
R³ represents 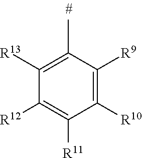
where
# represents the point of attachment to the triazinedione nitrogen atom,
R⁹ represents hydrogen,
R¹⁰ represents hydrogen, halogen or (C₁-C₄)-alkoxy,
R¹¹ represents (C₁-C₄)-alkyl, (C₁-C₄)-alkoxy or —N(R¹⁴R¹⁵),
where
R¹⁴ represents (C₁-C₄)-alkyl,
R¹⁵ represents hydrogen or (C₁-C₄)-alkyl,
or
R¹¹ represents 5- or 6-membered heterocyclyl, in which 5- or 6-membered heterocyclyl may be substituted by 1 or 2 sub stituents independently of one another selected from the group consisting of trifluoromethyl, (C₁-C₄)-alkyl and oxo,
R¹² represents hydrogen,
R¹³ represents hydrogen or (C₁-C₄)-alkyl,
or
R³ represents a group of the formula 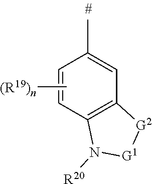
where
# represents the point of attachment to the triazinedione nitrogen atom,
G¹ represents C═O or SO₂,
G² represents CR^{21A}R^{21B}, NR²², O or S,
where
R^{21A} represents hydrogen, fluorine, (C₁-C₄)-alkyl or hydroxy,
R^{21B} represents hydrogen, fluorine, chlorine, (C₁-C₄)-alkyl or trifluoromethyl,
or
R^{21A} and R^{21B} together with the carbon atom to which they are attached form a 3- to 6-membered carbocycle,
R²² represents hydrogen, (C₁-C₆)-alkyl or (C₃-C₇)-cycloalkyl,
R¹⁹ represents fluorine or methyl,
n represents a number 0 or 1,
R²⁰ represents hydrogen, (C₁-C₆)-alkyl or (C₃-C₆)-cycloalkyl, and the salts, solvates and solvates of the salts thereof.

3. The compound of claim 1 of the formula (I) in which
R¹ represents hydrogen, methyl or ethyl,
R² represents a group of the formula 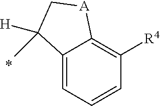
where
* represents the point of attachment to the triazinedione nitrogen atom,
A represents —CH₂— or —CH₂—CH₂—,
R⁴ represents chlorine or trifluoromethyl,
R³ represents 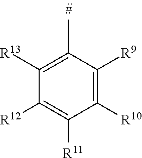
where
# represents the point of attachment to the triazinedione nitrogen atom,
R⁹ represents hydrogen,
R¹⁰ represents hydrogen,
R¹¹ represents methoxy or ethoxy,
or
R¹¹ represents a group of the formula 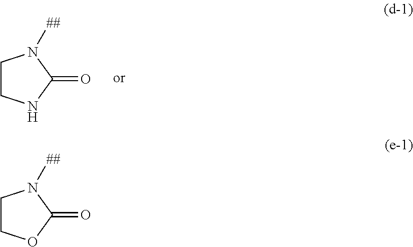
in which
## represents the point of attachment to the phenyl ring,
R¹² represents hydrogen,
R¹³ represents hydrogen or methyl,
or
R³ represents a group of the formula 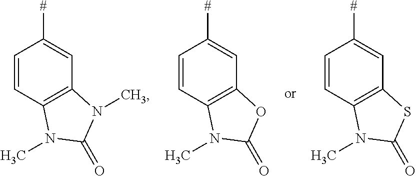
where
# represents the point of attachment to the triazinedione nitrogen atom, and the salts, solvates and solvates of the salts thereof.

4. A method of preparing the compound of claim 1, in which
[A] a compound of the formula (II)
H₂N—R³ (II)
in which
R³ is as defined above,
is diazotized in an inert solvent using sodium nitrite and a suitable acid to give a compound of the formula (II-1) 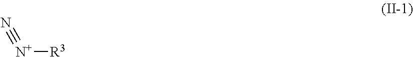
in which
R³ has the meaning given above,
and the diazonium salt is, optionally in the presence of a suitable base, reacted with a compound of the formula (III) 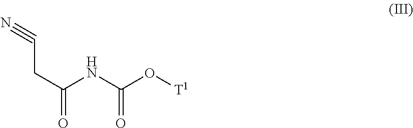
in which
T¹ represents (C₁-C₄)-alkyl,
to give a compound of the formula (IV) 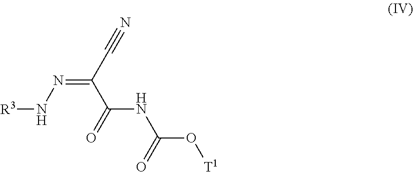
in which
R³ and T¹ each have the meanings given above, this is then converted in an inert solvent, optionally in the presence of a suitable base, into a compound of the formula (V) 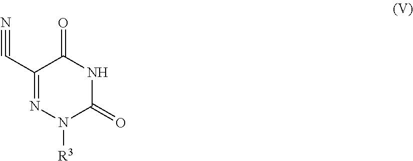
in which
R³ has the meaning given above,
subsequently reacted under Mitsunobu conditions with an activating agent, e.g. diethyl azodicarboxylate (DEAD) or diisopropyl azodicarboxylate (DIAD), and a phosphine reagent, e.g. triphenylphosphine or tributylphosphine, in an inert solvent with a compound of the formula (VI) 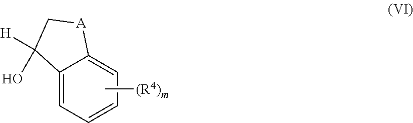
to give a compound of the formula (VII) 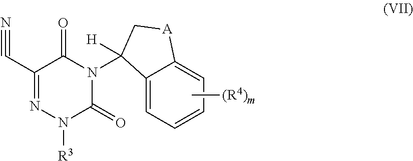
in which
A, m, R³ and R⁴ have the meanings given above, and this is then hydrolyzed in an inert solvent in the presence of a suitable acid or base to give a compound of the formula (I-1) 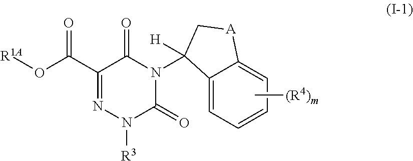
in which
A, m, R³ and R⁴ have the meanings given above, and
R^{1A} represents hydrogen,
or
[B] a compound of the formula (V) 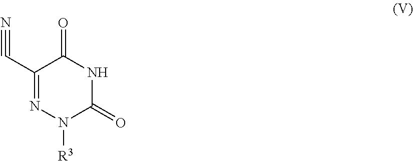
in which
R³ has the meaning given above,
is hydrolyzed in an inert solvent in the presence of a suitable acid or base to give a compound of the formula (VIII) 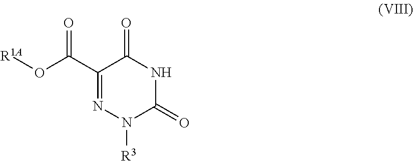
in which
R^{1A} represents hydrogen,
and
R³ has the meaning given above,
the acid function is then esterified to give a compound of the formula (IX) 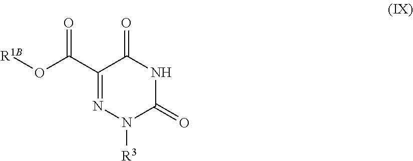
in which
R³ has the meanings given above,
and
R^{1B} represents (C₁-C₄)-alkyl,
and this is subsequently analogously to process [A] under Mitsunobu conditions with an activating agent, e.g. diethyl azodicarboxylate (DEAD) or diisopropyl azodicarboxylate (DIAD), and a phosphine reagent, e.g. triphenylphosphine or tributylphosphine, in an inert solvent with a compound of the formula (VI) 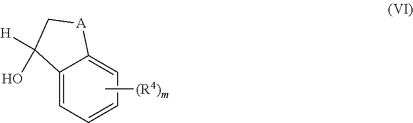
in which
A, m and R⁴ have the meanings given above, converted into a compound of the formula (I-2) 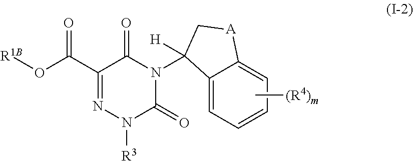
in which
A, m, R³ and R⁴ have the meanings given above,
and
R^{1B} represents (C₁-C₄)-alkyl,
or
[C] a compound of the formula (I-2) is hydrolysed in an inert solvent in the presence of a suitable acid or base to give a compound of the formula (I-1) 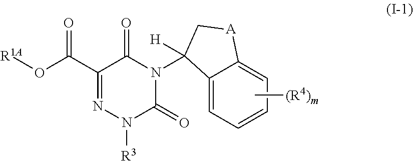
in which A, m, R³ and R⁴ each have the meanings given above,
and
R^{1A} represents hydrogen,
any protecting groups are detached and/or the compounds of the formulae (I-1) and (I-2) are, where appropriate, converted with the appropriate (i) solvents and/or (ii) bases or acids to the solvates, salts and/or solvates of the salts thereof.

5. A method for treatment and/or prophylaxis of diseases using the compound of claim 1.

6. A method for treatment and/or prophylaxis of heart failure, pulmonary hypertension, chronic obstructive pulmonary disease, asthma, kidney failure, nephropathy, fibrotic disorders of the internal organs and dermatological fibroses using the compound of claim 1.

7. A medicament for the treatment and/or prophylaxis of heart failure, pulmonary hypertension, chronic obstructive pulmonary disease, asthma, kidney failure, nephropathy, fibrotic disorders of the internal organs and dermatological fibroses comprising the compound of claim 1.

8. A medicament comprising the compound of claim 1 in combination with one or more inert, nontoxic, pharmaceutically suitable excipients.

9. A medicament comprising the compound of claim 1 in combination with one or more further active ingredients selected from the group consisting of calcium antagonists, angiotensin AII antagonists, ACE inhibitors, vasopeptidase inhibitors, endothelin antagonists, renin inhibitors, alpha-receptor blockers, beta-receptor blockers, mineralocorticoid receptor antagonists, rho-kinase inhibitors, diuretics, kinase inhibitors, matrix metalloprotease inhibitors, stimulators and activators of soluble guanylate cyclase and phosphodiesterase inhibitors.

10. A method of using the medicament of claim 8 for treatment and/or prophylaxis of heart failure, pulmonary hypertension, chronic obstructive pulmonary disease, asthma, kidney failure, nephropathy, fibrotic disorders of the internal organs and dermatological fibroses.

11. A method for treatment and/or prophylaxis of heart failure, pulmonary hypertension, chronic obstructive pulmonary disease, asthma, kidney failure, nephropathy, fibrotic disorders of the internal organs and dermatological fibroses in humans and animals using an effective amount of the compound of claim 8.

12. A method of using the medicament of claim 9 for treatment and/or prophylaxis of heart failure, pulmonary hypertension, chronic obstructive pulmonary disease, asthma, kidney failure, nephropathy, fibrotic disorders of the internal organs and dermatological fibroses.

13. A method for treatment and/or prophylaxis of heart failure, pulmonary hypertension, chronic obstructive pulmonary disease, asthma, kidney failure, nephropathy, fibrotic disorders of the internal organs and dermatological fibroses in humans and animals using an effective amount of the medicament of claim 9.
